(19) United States
(12) Patent Application Publication
(10) Pub. No.: US 2004/0009166 A1
(43) Pub. Date: Jan. 15, 2004

(54) Single chain antigen-binding polypeptides for polymer conjugation

(76) Inventors: David R. Filpula, Piscataway, NJ (US); Karen Yang, Edison, NJ (US); Amartya Basu, Berkley Heights, NJ (US); Maoliang Wang, East Brunswick, NJ (US)

Correspondence Address: MUSERLIAN, LUCAS & MERCANTI, LLP, 600 THIRD AVENUE, NEW YORK, NY, 10016, US

(21) Appl. No.: 10/423,847

(22) Filed: Apr. 25, 2003

Related U.S. Application Data
(63) Continuation-in-part of application No. 09/791,578, filed on Feb. 26, 2001.
Application No. 09/791,578 is a continuation of application No. 09/069,842, filed on Apr. 30, 1998, now abandoned.
(60) Provisional application No. 60/044,449, filed on Apr. 30, 1997.
(60) Provisional application No. 60/050,472, filed on Jun. 23, 1997.
(60) Provisional application No. 60/063,074, filed on Oct. 27, 1997.
(60) Provisional application No. 60/067,341, filed on Dec. 2, 1997.

(51) Int. Cl. A61K 39/395; C07K 16/46

(52) U.S. Cl. 424/130.1; 530/387.1; 424/178.1

(57) ABSTRACT

The present invention relates to monovalent and multivalent single-chain antigen-binding polypeptides with site-specific modifications. The provided polypeptides are capable of being covalently linked or conjugated to polyalkylene oxides at the modified sites. The resulting conjugates retain antigen binding properties and exhibit prolonged circulating time and reduced antigenicity relative to unconjugated single chain antigen binding polypeptides. Methods and compositions for making and using the single chain antigen-binding polypeptides with site-specific modifications are also provided.

This application claims priority from copending U.S. application Ser. Nos. 09/791,578, filed on Feb. 26, 2001, and 09/791,540, also filed on Feb. 26, 2001, that are continuations of U.S. application Ser. No. 09/069,842, filed on Apr. 30, 1998, now abandoned, and that in turn claimed priority of U.S. Ser. Nos. 60/044,449 filed Apr. 30, 1997; 60/050,472 filed Jun. 23, 1997; 60/063,074 filed Oct. 27, 1997; and 60/067,341 filed Dec. 2, 1997, each of which is incorporated herein by reference herein in its entirety.

### BACKGROUND OF THE INVENTION

1. Field of The Invention

The present invention relates to monovalent and multivalent single-chain antigen-binding polypeptides with site-specific modifications facilitating site-specific covalent linkage of polymers to the inventive polypeptides. The invention provides such modified single-chain antigen-binding polypeptides, vectors and host cells encoding the same, as well as methods of making and using the polypeptides. The invention also provide for conjugates of the modified single-chain antigen-binding polypeptides with polymers, such as polyalkylene oxide (“PAO”) to provide new and improved prodrugs, and methods of making and using these conjugates.

2. Description of the Related Art

Naturally occurring antibodies are immunoglobulins produced by the immune system of vertebrates, including mammals, in response to the presence of one or more specific substances, i.e., antigens, when these are recognized as foreign by the immune cells of the animal. In humans, there are five classes of antibodies which have the ability to selectively recognize and preferentially bind to specific antigens. Each antibody class has the same basic structure or multiples of that structure. The basic unit consists of two identical polypeptides called heavy or H chains (molecular weight in IgG approximately 50,000 Daltons each) and two identical polypeptides called light or L chains (molecular weight approximately 25,000 Daltons each). Each of the five antibody classes has a similar set of light chains and a distinct set of heavy chains. A light chain is composed of one variable and one constant domain, while a heavy chain is composed of one variable and three or more constant domains. The variable domains determine the specificity of the immunoglobulin, the constant regions have other functions.

Broadly, pairs of suitable light and heavy polypeptide chains are associated in natural antibodies, and in other types of antibodies, to form antigen binding sites. Each individual light and heavy chain folds into regions of approximately 110 amino acids, assuming a conserved three-dimensional conformation. The light chain comprises one variable region (V_{L}) and one constant region (C_{L}), while the heavy chain comprises one variable region (V_{H}) and three constant regions (C_{H1}, C_{H2} and C_{H3}). Pairs of regions associate to form discrete structures. In particular, the light and heavy chain variable regions associate to form an “Fv” area which contains the antigen-binding site. The constant regions are not necessary for antigen binding and in some cases can be separated from the antibody molecule by proteolysis, yielding biologically active (i.e., binding) variable regions composed of half of a light chain and one quarter of a heavy chain.

Further, all antibodies of a certain class and their Fab fragments (i.e., fragments composed of V_{L}, C_{L}, V_{H}, and C_{H1}) whose structures have been determined by x-ray crystallography show similar variable region structures despite large differences in the sequence of hypervariable segments even when from different animal species. The immunoglobulin variable region seems to be tolerant towards mutations in the antigen-binding loops. Therefore, other than in the hypervariable regions, most of the so-called “variable” regions of antibodies, which are defined by both heavy and light chains, are, in fact, quite constant in their three dimensional arrangement. See for example, Huber, R., Science 233:702-703 (1986), incorporated by reference herein.

Natural antibodies are typically heterogenous, binding to many different epitopes, or parts of a foreign antigen. In contrast, monoclonal antibodies (“MAbs”) are antibodies that are homogenous in their binding affinity. MAbs have been shown to be useful both as diagnostic and therapeutic agents. MAbs are produced routinely by established procedures, e.g., from hybridomas generated by fusion of mouse lymphoid cells with an appropriate mouse myeloma cell line, as well as by more advanced recombinant techniques.

Even smaller antibody-like proteins or polypeptides are formed of antigen binding sites with minimal additional structure. These are art-known as single-chain antigen-binding proteins or polypeptides (“SCAs”) or single-chain variable fragments of antibodies (“sFv”). These may incorporate a linker polypeptide to bridge individual variable regions, V_{L} and V_{H}, into a single polypeptide chain.

A description of the theory and production of single-chain antigen-binding proteins is found in Ladner et al., U.S. Pat. Nos. 4,946,778, 5,260,203, 5,455,030 and 5,518,889, and in Huston et al., U.S. Pat. No. 5,091,513 (“biosynthetic antibody binding sites” (BABS)), which disclosures are all incorporated herein by reference. The single-chain antigen-binding proteins produced under the process recited in the above patents have binding specificity and affinity substantially similar to that of the corresponding Fab fragment.

Broadly, pairs of suitable light and heavy chains can be associated to form antigen binding sites. Each individual light and heavy chain folds into regions of approximately 110 amino acids, assuming a conserved three-dimensional conformation. The light chain comprises one variable region (V_{L}) and one constant region (C_{L}), while the heavy chain comprises one variable region (V_{H}) and three constant regions (C_{H1}, C_{H2} and C_{H3}). Pairs of regions associate to form discrete structures. In particular, the light and heavy chain variable regions associate to form an “Fv” area which contains the antigen-binding site. The constant regions are not necessary for antigen binding and in some cases can be separated from the antibody molecule by proteolysis, yielding biologically active (i.e., binding) variable regions composed of half of a light chain and one quarter of a heavy chain.

Further, x-ray crystallography has confirmed that all antibodies of a particular class, and their Fab fragments (i.e., fragments composed of V_{L}, C_{L}, V_{H}, and C_{H1}) show similar variable region structure, but large differences in the sequences of their respective hypervariable segments. This is also observed in comparisons of antibodies derived from different respective animal species. The immunoglobulin variable region seems to be tolerant of mutations in the antigen-binding loops. Therefore, other than in the hypervariable regions, most of the so-called “variable” regions of antibodies, which are defined by both heavy and light chains, are, in fact, quite constant in their three dimensional arrangement. See for example, Huber, R., Science 233:702-703 (1986), incorporated by reference herein.

The in vivo properties of SCA polypeptides are different from those of MAbs and larger, more conventional antibody fragments. Their small size allows SCas to be cleared more rapidly from the blood, and to penetrate more rapidly into tissues (Milenic, D. E. et al., Cancer Research 51:6363-6371(1991); Colcher et al., J. Natl. Cancer Inst. 82:1191 (1990); Yokota et al., Cancer Research 52:3402 (1992)). In addition, SCA polypeptides are not retained in tissues such as the liver and kidneys due to the absence of a constant region normally present in antibody molecules. Thus, SCA polypeptides have applications in cancer diagnosis and therapy, where rapid tissue penetration and clearance are advantageous.

Synthetic antigen binding proteins are also described by Huston et al. in U.S. Pat. No. 5,091,513, incorporated by reference herein. The described proteins are characterized by one or more sequences of amino acids constituting a region that behaves as a biosynthetic antibody binding site (BABS). The sites comprise (1) noncovalently associated or disulfide bonded synthetic V_{H} and V_{L} regions, (2) V_{H}—V_{L} or V_{L}—V_{H} single chains wherein the V_{H} and V_{L} are attached to a polypeptide linker, or (3) individual V_{H} or V_{L} domains. The binding domains comprises complementarity determining regions (CDRs) linked to framework regions (FRs), which may be derived from separate immunoglobulins. It should be noted that the Huston et al. proteins include one characterized by having an initial heavy chain, i.e., the V_{H}—peptide linker—V_{L} domain.

Multivalent antigen-binding proteins are known. As described herein, a multivalent antigen-binding protein includes two or more single-chain protein molecules. These can be associated or linked by covalent or noncovalent bonding. Enhanced antigen binding activity, di- and multi-specific binding, and other novel uses of multivalent antigen-binding proteins have been demonstrated. See, e.g., Whitlow, M., et al., Protein Engng. 7:1017-1026 (1994); Hoogenboom, H. R., Nature Biotech. 15:125-126(1997); and WO 93/11161, and co-owned U.S. Pat. Nos. 5,869,620, 6,025,165, 6,027,725, 6,103,889, 6,121,424 and 6,515,110, all incorporated by reference herein.

Although peptides, such as the single-chain polypeptides described above, and fusion proteins thereof, have not been associated with significant antigenicity in mammals, it has been desirable to prolong the circulating life and even further reduce the possibility of an antigenic response.

One way to enhance the circulating life and reduce the antigenicity of proteins and polypeptides has been to conjugate them to polymers, such as polyalkylene oxides. However, the relatively small size of the polypeptides and their delicate structure/activity relationship, have made polyethylene glycol modification difficult and unpredictable.

To effect covalent attachment of polyalkalene oxides to a protein, the hydroxyl end groups of the polymer must first be converted into reactive functional groups. This process is frequently referred to as “activation” and the product is called “activated PEG” or activated polyalkylene oxide. For example, methoxy poly(ethylene glycol) (mPEG), capped on one end with a functional group, reactive towards amines on a protein molecule, is used in most cases.

A number of activated polymers, such as succinimidyl succinate derivatives of PEG (“SS-PEG”), have been introduced (Abuchowski et al., Cancer Biochem. Biophys. 7:175-186 (1984)). SS-PEG reacts quickly with proteins (30 minutes) under mild conditions yielding active yet extensively modified conjugates. Zalipsky, in U.S. Pat. No.5,122,614, discloses poly(ethylene glycol)-N-succinimide carbonate and its preparation. This form of the polymer is said to react readily with the amino groups of proteins, as well as low molecular weight peptides and other materials that contain free amino groups. Other linkages between the amino groups of the protein, and the PEG are also art known such as urethane linkages (Veronese et al., Appl. Biochem. Biotechnol. 11:141-152 (1985)), carbamate linkages (Beauchamp et al., Analyt. Biochem. 131:25-33 (1983)), and others.

However, despite these and other methods, it has often been found that the resulting conjugates lack sufficient retained activity. For example, Benhar et al. (Bioconjugate Chem. 5:321-326 (1994)) observed that PEGylation of a recombinant single-chain immunotoxin resulted in the loss of specific target immunoreactivity of the immunotoxin. The loss of activity of the immunotoxin was the result of PEG conjugation at two lysine residues within the antibody-combining region of the immunotoxin. To overcome this problem, Benhar et al. replaced these two lysine residues with arginine residues and were able to obtain an active immunotoxin that was 3-fold more resistant to inactivation by derivatization.

Another suggestion for overcoming these problems discussed above is to use longer, higher molecular weight polymers. These materials, however, are difficult to prepare and expensive to use. Further, they provide little improvement over more readily available polymers. Another alternative suggested is to attach two strands of polymer via a triazine ring to amino groups of a protein. See, for example, Enzyme 26:49-53 (1981) and Proc. Soc. Exper. Biol. Med., 188:364-369 (1988). However, triazine is a toxic substance that is difficult to reduce to acceptable levels after conjugation.

An examination of the three-dimensional structure of an SCA protein reveals that the C-terminus and the linker region are farthest removed from the antigen-binding site and therefore might be sites for polymer conjugation wherein the attached polymer does not sterically block or disrupt the conformation of the antigen-binding site or the surrounding Fv architecture (Wang M., et al., 1998 Protein Engng 11: 1277-1283) in the context of positioning sites for polymer linkage to inserted residues that are glycosylated, in vivo, during production of those proteins.

Efforts to position amino acid residues within SCA structure for more effective polymer conjugation have been described by the following co-owned parents of the present patent application, all of which are incorporated by reference herein: U.S. Ser. Nos. 09/791,578 and 09/791,540 both filed on Feb. 26, 2001, describing selectively positioned Cys and oligo Lys residues.

Co-owned U.S. Ser. Nos. 09/956,087 and 09/956,086, both Sep. 20, 2001 describe tandem and triplet ASN, and related sites in an SCA for selectively positioned glycosylation to which polymers are selectively conjugated. However, there remains a need in the art for further options and improvements in positional conjugation of polymers to SCA proteins, as well as further options and improvements in conjugation chemistry, that allows for the binding activity and specificity of the polypeptide to be retained, along with all of the benefits of polymer conjugations.

### SUMMARY OF THE INVENTION

In order to address these longstanding needs, the invention provides for a single-chain antigen-binding polypeptide capable of site-specific conjugation to a polyalkylene oxide polymer, that comprises,

a first polypeptide comprising an antigen-binding portion of a variable region of an antibody heavy or light chain;

a second polypeptide comprising an antigen-binding portion of a variable region of an antibody heavy or light chain; and

a peptide linker linking the first and second polypeptides,

wherein the single-chain antigen-binding polypeptide has at least one Cys residue which is capable of being conjugated to a polyalkylene oxide polymer, and has at least one antigen binding site. The Cys residue is preferably located at one or more of the following positions.:

a C-terminus of the heavy chain or light chain variable region;

an N-terminus of the heavy chain or light chain variable region;

any amino acid position of the peptide linker;

both the N-terminus and C-terminus;

position 2 of the linker;

both position 2 of the linker and the C-terminus; and

combinations thereof.

More preferred Cys positions include, e.g., position 2 of the linker, the C-terminus and combinations thereof. The C-terminus is preferably a naturally occuring C-terminus, but can also include any art known modifications thereof.

The SCA of the invention is optionally formed of variable regions from light and/or heavy chains of an antibody of interest.

The invention also provides conjugates comprising the inventive single-chain antigen-binding polypeptides or proteins, wherein the conjugates include a substantially non-antigenic polymers, e.g., a polyalkylene oxide polymer. The polyalkylene oxide is preferably a polyethylene glycol or “PEG” polymer.

The polyalkylene oxide is any suitable size range, but preferably ranges in size from about 5,000 to about 40,000 Daltons.

Preferably the polyalkylene oxide polymer is covalently linked to the single-chain antigen-binding polypeptide at a Cys residue.

Preferably, the polyalkylene oxide is linked to the single-chain antigen-binding polypeptide at a Cys residue via a linker, such as, for instance, a maleimide, vinylsulfone, thiol, orthopyridyl disulfide and/or a iodoactemide linker. The maeleimide linker is most preferred.

In the conjugates of the invention, the polyalkylene oxide is optionally conjugated to at least two single-chain antigen-binding polypeptides, wherein each single-chain antigen-binding polypeptide is the same, or different.

Optionally, the conjugate, e.g. either the PAO or the SCA, or both, is further linked or conjugated to an additional functional moiety, e.g., a detectable label or tag.

The invention also provides for polynucleotides encoding the single-chain antigen-binding polypeptides, replicable expression vectors comprising the polynucleotides, and suitable host cells for expressing the same.

The inventive SCA proteins are produced by any suitable, art-known process or method, but are preferably produced, as exemplified herein, by culturing a host cell comprising an SCA encoding expression vector and collecting the single-chain antigen-binding polypeptide expressed by the host cell.

Methods for using the inventive SCAs and polymer conjugates are also provided. Simply by way of example, one such methods includes the steps of:

contacting the sample with a reagent comprising the single-chain antigen-binding detecting whether the single-chain antigen-binding polypeptide has bound to the antigen.

A method of detecting an antigen suspected of being in a sample, employing the inventive SCAs and/or polymer conjugates, includes the steps of:

contacting the sample with a reagent comprising an SCA conjugate of the invention, and wherein the polypeptide is conjugated to a polyalkylene oxide polymer and detecting whether the conjugate or the single-chain antigen-binding polypeptide has bound to the antigen.

For all of the above methods, the conjugate, either the SCA or the polylmer, is optionally anchored to a solid substrate.

Also provided are methods of treating or diagnosing a disease or disorder in a mammal in need thereof, comprising administering an effective amount of the single-chain antigen-binding polypeptide of the invention, wherein the single-chain antigen-binding polypeptide binds to an antigen that is related to treating or diagnosing the disease or disorder.

The invention further provides for a protein comprising two or more single-chain antigen-binding polypeptides capable of site-specific conjugation to at least one polyalkylene oxide polymer, wherein each single-chain antigen-binding polypeptide comprises:

a first polypeptide comprising an antigen binding portion of a variable region of an antibody heavy or light chain;

a second polypeptide comprising an antigen binding portion of a variable region of

an antibody heavy or light chain; and

a peptide linker linking the first and second polypeptides,

wherein at least one of the single-chain antigen-binding polypeptides has from 1 to 4 Cys residues capable of being conjugated to a polyalkylene oxide polymer, and wherein the Cys residue is located at a position of each Cys-bearing single-chain antigen-binding polypeptide that includes one or more of the following:

a C-terminus of the heavy chain or light chain variable region;

an N-terminus of the heavy chain or light chain variable region;

any amino acid position of the peptide linker;

both the N-terminus and C-terminus;

position 2 of the linker;

both position 2 of the linker and the C-terminus; and combinations thereof;

wherein each single-chain antigen-binding polypeptide is the same or different, and the protein is optionally bivalent or trivalent (meaning that it can optionally be designed to bind to more than one antigen, wherein each antigen that is bindable is the same or different..

The inventive multivalent protein is prepared by art-known methods wherein each SCA is linked by covalent or noncovalent linkers, e.g., peptide linkers, disulfide linkers, and the like. In an alternative option, the protein is assembled with noncovalent linkers by reducing and refolding the constituent SCA polypeptides. In the later option, it is particularly preferred that the peptide linker of the constituent single-chain antigen-binding proteins or polypeptides range in size from 2 to 18 residues.

The invention further provides for a multivalent protein, having the particular Cys residues in one or more of the constituent SCA polypeptides, that is encoded as a single, multivalent protein. A polynucleotide encoding such a single chain multivalent protein is also contemplated as part of the present invention.

### BRIEF DESCRIPTION OF THE DRAWINGS

FIG. 1A illustrates the sequence of the DNA molecule encoding SCA 2-7-SC-1 (SEQ ID NO: 1) having the construction V_{L}-218-V_{H}-his₆ and no Cys mutein, and the expressed protein (SEQ ID NO: 10).

FIG. 1B illustrates the sequence of the DNA molecule encoding 2-7-SC-2 (SEQ ID NO: 2) having the construction V_{L}-218-V_{H}-his₆ and that encodes an SCA with a C-terminus Cys, and the expressed protein (SEQ ID NO: 11).

FIG. 1C illustrates the sequence of the DNA molecule encoding 2-7-SC-3 (SEQ ID NO: 3) having the construction V_{H}-(GGGGS)₃-V_{L}-his₆ and that encodes an SCA with a C-terminus Cys, and the expressed protein (SEQ ID NO: 12).

FIG. 1D illustrates the sequence of the DNA molecule encoding 2-7-SC-4 (SEQ ID NO: 4) having the construction V_{L}-218-V_{H} and that encodes an SCA with a C-terminus Cys, and the expressed protein (SEQ ID NO: 13).

FIG. 1E illustrates the sequence of the DNA molecule encoding 2-7-SC-5 (SEQ ID NO: 5) having the construction V_{L}-218-V_{H}-his₆ and that encodes an SCA with a Cys at linker position 2, and the expressed protein (SEQ ID NO: 14).

FIG. 1F illustrates the sequence of the DNA molecule encoding 2-7-SC-6 (SEQ ID NO: 6) having the construction V_{L}-218-V_{H}-his₆ and that encodes an SCA with a Cys at linker position 2 and a Cys at the C terminus, and the expressed protein (SEQ ID NO: 15).

FIG. 1G illustrates the sequence of the DNA molecule encoding 2-7-SC-7 (SEQ ID NO: 7) having the construction V_{H}-(GGGGS)₃-V_{L}-his₆ and that encodes an SCA with a Cys at linker position 2, and the expressed protein (SEQ ID NO: 16).

FIG. 1H illustrates the sequence of the DNA molecule encoding 2-7-SC-8 (SEQ ID NO: 8) having the construction V_{L}-218-V_{H}-his₆ and that encodes an SCA with a Cys at both the N-terminus and C-terminus, and the expressed protein (SEQ ID NO: 17).

FIG. 1-I illustrates the sequence of the DNA molecule encoding 2-7-SC-9 (SEQ ID NO: 9) having the construction V_{L}-GGGGS-V_{H}-his₆ and that encodes an SCA with no free Cys, and the expressed protein (SEQ ID NO: 18).

FIG. 2A. illustrates clone 2-7-SC-2 SCA expression. Expression of the SCA protein is induced by 1% methanol or MeOH in Pichia culture. 27 kDa is marked by the arrow (“→”)

FIG. 2B illustrates expression and purification data for clone 2-7-SC-2, including the SDS-PAGE gel analysis by Coomassie Blue staining of the fractions, and the yield at each step. A small amount of ˜54 kDa disulfide-linked dimer is visible in the stained gel. Legend: STD, Mark12 protein molecular weight standards; SUP, fermentation harvest supernatant; DIA, diafiltered supernatant; DEAE, first DEAE chromatography flow-through; Ni⁺⁺, eluted sample after nickel affinity chromatography; DEAE, second DEAE chromatography. Peaks are visible at about 27 kDa, as identified by the carrot “>”.

FIG. 3A illustrates the structure of mPEG-MAL.

FIG. 3B illustrates the structure of mPEG₂(MAL).

FIG. 3C illustrates the structure of mPEG(MAL)₂.

FIG. 3D illustrates the structure of mPEG₂(MAL)₂.

FIG. 3E illustrates reaction of activated PEG-MAL with a thiol-SCA.

FIG. 3F illustrates a vinylsulfone active PEG.

FIG. 4 is a spectrograph plot of absorbance verses wavelength between 200 and 400 nm. Curve A is cysteine, 3 mM; curve B is PEG-MAL (1 mM)+cysteine (3 mM) post-reaction; and curve C is PEG-MAL 1 mM.

FIG. 5A illustrates an SDS-PAGE analysis of 2-7-SC-5 and 2-7-SC-5 conjugates with visualization by Brilliant Blue Stain. Lane 1 provides MARK12 (Invitrogen) protein size standards, lane 2 is non-conjugated 2-7-SC-5 SCA, lane 3 is PEG(20K)2-7-SC-5 SCA and lane4 is PEG(40K)2-7-SC-5 SCA.

FIG. 5B illustrates an SDS-PAGE analysis of 2-7-SC-5 and 2-7-SC-5 conjugates with visualization by iodine staining. Lane 1 provides MARK12 protein size standards, lane 2 is non-conjugated 2-7-SC-5 SCA, lane 3 is PEG(20K)2-7-SC-5 SCA and lane4 is PEG(40K)2-7-SC-5 SCA.

FIG. 6A illustrates flow cytometry analysis of the binding of biotinylated TNFα to cell receptor in the presence of 2-7-SC-2 SCA. Curve 1 represents the cell population without fluorescence labeling, curve 2 represents the cell population after binding to biotin-TNFα and then to streptavidin-PE, curve 3 represents the cell population preincubated with SCA, biotin-TNFα, and then streptavidin-PE.

FIG. 6B illustrates flow cytometry analysis of the binding of biotinylated TNFα to cell receptor in the presence of 2-7-SC-2 PEG(20K) SCA. Curve 1 represents the cell population without fluorescence labeling, curve 2 represents the cell population after binding to biotin-TNFα and then to streptavidin-PE, curve 3 represents the cell population preincubated with PEG-SCA, biotin-TNFα and then to streptavidin-PE.

FIG. 6C illustrates flow cytometry analysis of the binding of biotinylated TNFα to cell receptor in the presence of 2-7-SC-2 PEG(40K) SCA. Curve 1 represents the cell population without fluorescence labeling, curve 2 represents the cell population after binding to biotin-TNFα and then to streptavidin-PE, curve 3 represents the cell population preincubated with PEG-SCA, TNFα and then to streptavidin-PE.

FIG. 7 shows a Western blot analysis of D2E7 2-7-SC-2 SCA protein and PEG-SCA derivatives. The primary detection antibody was anti-2-7-SC-1 SCA rabbit antiserum prepare from rabbits immunized with the purified recombinant SCA protein. Lane 1 and 7, molecular weight markers (250, 148, 98, 64, 50, 36, 22, 16, 6 and 4 kDa); lane 2, 2-7-SC-2 SCA protein; lane 3, ethyl-2-7-SC-2 ; lane 4, PEG (5 kDa)-2-7-SC-2 ; lane 5, PEG (20 kDa)-2-7-SC-2 ; lane 6, PEG (40 kDa)-2-7-SC-2.

FIG. 8 shows the scanned image intensity of the bands of D2E7 2-7-SC-2 and PEGylated forms confirming reactivity of this anti-D2E7 antiserum with the recombinant SCA proteins and PEG-SCA conjugates. Band A is 2-7-SC-2, Band B is 2-7-SC-5, Band C is PEG(20k)-2-7-SC-2, Band D is PEG(20k)-2-7-SC-5, Band E is PEG(40k)-2-7-SC-2, Band F is PEG(40k)-2-7-SC-5.

FIG. 9 shows the SDS PAGE analysis of a representative set of samples for the pharmacokinetic studies. 2-7-SC-2 SCA proteins and 2-7-SC-2 PEG-SCA conjugates were examined on Coomassie Blue stained gels. On the left gel, the loaded samples were non-reduced. On the right gel, the samples were reduced with 3 mM beta-mercaptoethanol and heated to 85° C. for 2 minutes prior to loading. Approximately ten micrograms of protein was loaded for each lane. Legend: MM—molecular weight standards; lane 1, 2-7-SC-2 SCA; lane 2, 2-7-SC-2 SCA modified with N-ethylmaleimide; lane 3, 2-7-SC-2 SCA-PEG(40 kDa); lane 4, 2-7-SC-2 SCA-PEG(20 kDa).

### DETAILED DESCRIPTION OF THE INVENTION

Accordingly, the present invention provides improved single-single chain antigen-binding polypeptides with engineered functional groups selected to facilitate site-directed conjugation to polymers, e.g., substantially non-antigenic polymers. Conjugates of the inventive polypeptides with polymers, as well as methods of making and using the same, are also provided.

The invention broadly relates to the discovery that single-chain antigen-binding proteins (“SCA”) or single-chain variable fragments of antibodies (“sFv”), have enhanced properties when conjugated to suitable polyalkylene oxide polymers at specific locations on the SCA polypeptide. The SCAs of the invention are engineered to include functional groups for selective polymer conjugation at such specific locations. The benefits of polymer conjugation broadly include substantially reduced antigenicity in vivo, and increased circulating half-life after administration to an animal or human patient. Without meaning to be bound by any theory or hypothesis as to how the inventive SCAs provide conjugates with desirable properties, it is believed that, by engineering the binding sites at selected locations on the polypeptide chain or chains, interference with antigen binding function and chain tertiary structure by the presence of the conjugated polymer(s) is avoided or minimized.

In order to better appreciate the scope of the invention, the following terms are defined. The terms “single-chain antigen-binding molecule” (SCA) or “single-chain Fv” (sFv) are used interchangeably herein unless otherwise specified. The terms, “protein” and “polypeptide” are also used interchangeably unless otherwise specified. Broadly, an SCA is structurally defined as comprising the binding portion of a first polypeptide from the variable region of an antibody V_{L} (or V_{H}), associated with the binding portion of a second polypeptide from the variable region of an antibody V_{H} (or V_{L}), the two polypeptides being joined by a peptide linker linking the first and second polypeptides into a single polypeptide chain, such that the first polypeptide is N-terminal to the linker and second polypeptide is C-terminal to the first polypeptide and linker.

The SCA thus comprises a pair of variable regions connected by a polypeptide linker. The regions may associate to form a functional antigen-binding site, as in the case wherein the regions comprise a light-chain and a heavy-chain variable region pair with appropriately paired complementarity determining regions (CDRs). In this case, the single-chain protein is broadly referred to as a “single-chain antigen-binding protein” or “single-chain antigen-binding molecule” or “single-chain antigen-binding polypeptide.” As defined above, the SCAs are optionally “monovalent” or “multivalent.” Monovalent SCAs are engineered to include only a single antigen binding site, i.e., a single pair of variable regions connected by a polypeptide linker associating to form the antigen binding site. Multivalent SCAs are antigen binding proteins engineered to include two or more antigen binding sites, ie., two or more pairs of variable regions connected by a polypeptide linker, including SCAs that include two or more single-chain antigen-binding polypeptides as described above. The constituent SCA moieties are associated by any art-known method.

In one embodiment, multivalent binding proteins according to the invention include two or more SCAs that are noncovalently associated so as to remain fully functional as antigen binding proteins. In another embodiment, multivalent binding proteins include two or more SCAs that are associated by covalent linkage, e.g., via one of several art-known peptide or non-peptide linker chemistries. Further, multivalent binding proteins, e.g., formed of plural SCAs, can be expressed or synthetically constructed as a single peptide chain, analogously to a monovalent SCA, but with two or more repeated SCA domains, that are the same or different.

SCAs are constructed so that the V_{L} is the N-terminal domain followed by the linker and V_{H} (a V_{L}-Linker-V_{H} construction). In an alternative embodiment, SCAs are constructed so that V_{H} is the N-terminal domain followed by the linker and V_{L} (V_{H}-Linker-V_{L} construction). The preferred embodiment contains V_{L} in the N-terminal domain (see, Anand, N. N., et al., J. Biol. Chem. 266:21874-21879 (1991)). Optionally, multiple linkers are employed.

A description of the theory and production of single-chain antigen-binding proteins is found in Ladner et al., U.S. Pat. Nos. 4,946,778, 5,260,203, 5,455,030 and 5,518,889, and in Huston et al., U.S. Pat. No. 5,091,513 (“biosynthetic antibody binding sites” (BABS)), all incorporated herein by reference. The SCAs produced according to the above patents have binding specificity and affinity substantially similar to that of the corresponding Fab fragment.

Variable Domains (Fv)

The SCAs of the invention are constructed with variable domains (“Fv”) that are selected, derived or modeled from any desirable natural or artificial antibody. In another preferred embodiment, Fv for use in the invention are obtained from libraries of Fvs configured as permutation libraries, screened against a desired binding target(s). Simply by way of example, large numbers of MAbs have been employed by the art to obtain Fv domains and it is contemplated that Fv domains can be obtained, and employed in the SCAs of the invention, from any of these. Simply by way of example, and without limitation, the following MAbs are employed to provide Fv domains:

26-10, MOPC 315, 741F8, 520C9, McPC 603, D1.3, murine phOx, human phOx, RFL3.8 sTCR, 1A6, Sel55-4, 18-2-3, 4-4-20, 7A4-1, B6.2, CC49, 3C2, 2c, MA-15C5/K₁₂G₀, Ox, etc. (See, Huston, J. S. et al., Proc. Natl. Acad. Sci. (USA) 85:5879-5883 (1988); Huston, J. S. et al., SIM News 38(4) (Supp.):11 (1988); McCartney, J. et al., ICSU Short Reports 10:114 (1990); McCartney, J. E. et al., unpublished results (1990); Nedelman, M. A. et al., J. Nuclear Med. 32 (Supp.):1005 (1991); Huston, J. S. et al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci. (USA) 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. (USA) 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad. Sci. (USA) 87:9491-9494 (1990); Batra, J. K. et al., Mol. Cell. Biol. 11:2200-2205 (1991); Brinkmann, U. et al., Proc. Natl. Acad. Sci. (USA) 88:8616-8620 (1991); Seetharam, S. et al., J. Biol. Chem. 266:17376-17381 (1991); Brinkmann, U. et al., Proc. Natl. Acad. Sci. (USA) 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. (USA) 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Bio/Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. (USA) 88:8646-8650 (1991); Traunecker, A. et al., EMBO J. 10:3655-3659 (1991); Hoo, W. F. S. et al., Proc. Natl. Acad. Sci. (USA) 89:4759-4763 (1993)). All of the foregoing citations are incorporated by reference herein.

In particular, the anti-TNFα MAb described as D2E7 by U.S. Pat. No. 6,258,562, and the anti-erbB-2 MAb (HERCEPTIN™) described by Carter P et al., 1992, Proc Natl Acad Sci (USA) 89: 4285-4289 served as exemplary models for the engineering of SCAs and SCA-polyalkylene oxide conjugates, according to the invention. In addition, the CC49 MAb was developed by Dr. Jeffrey Schlom's group, Laboratory of Tumor Immunology and Biology, National Cancer Institute. It binds specifically to the pan-carcinoma tumor antigen TAG-72. See Muraro, R. et al., Cancer Research 48: 4588-4596 (1988). The anti-TAG-72 CC-49 SCA described by Filpula et al. 1996 (Antibody Engineering: A Practical Approach,, Oxford University Press, pp 253-268), was also prepared as a Cys-modified SCA and as an exemplary conjugate according to the invention. All of the foregoing citations are incorporated by reference herein.

Peptide Linkers

SCAs according to the invention include peptide linkers designed to span the C-terminus of V_{L}, or neighboring site thereof, and the N-terminus of V_{H}, or neighboring site thereof, or to link the C-terminus of V_{H} and the N-terminus of V_{L}.

The artisan will appreciate that linker length depends upon the nature of the polypeptides to be linked and the desired activity of the linked fusion polypeptide resulting from the linkage. Generally, the linker should be long enough to allow the resulting linked fusion polypeptide to properly fold into a conformation providing the desired biological activity, i.e., antigen-binding. In each particular case, the preferred length will depend upon the nature of the polypeptides to be linked and the desired activity of the linked fusion polypeptide resulting from the linkage.

Where conformational information is available, as is the case with the SCA polypeptides discussed below, the appropriate linker length may be estimated by consideration of the 3-dimensional conformation of the substituent polypeptides and the desired conformation of the resulting linked fusion polypeptide. Where such information is not available, the appropriate linker length may be empirically determined by testing a series of linked fusion polypeptides with linkers of varying lengths for the desired biological activity. Such linkers are described in detail in WO 94/12520, incorporated herein by reference.

Peptide linkers used to construct SCA polypeptides generally range in size from about 2 to about 50 amino acid residues in length, and preferably, from about to 2 to about 10 residues. In certain other embodiments, the linkers range in size from about 10 to about 30 residues. In certain more preferred embodiment, particularly for embodiments related to multivalent binders comprising two or more noncovalently associated SCA polypeptides, it is preferred that the linker range in size from about 2 to about 20 amino acid residues. More preferably, such linkers are serine rich or glycine rich.

The linkers are designed to be flexible, and it is recommended that an underlying sequence of alternating Gly and Ser residues be used. To enhance the solubility of the linker and the single chain Fv protein associated therewith, three charged residues may be included, two positively charged lysine residues (K) and one negatively charged glutamic acid residue (E). Preferably, one of the lysine residues is placed close to the N-terminus of V_{H}, to replace the positive charge lost when forming the peptide bond of the linker and the V_{H}. Such linkers are described in detail by co-owned U.S. Pat. No. 5,856,456, incorporated by reference herein. See also, Whitlow, M., et al., Protein Engng. 7:1017-1026 (1994), incorporated by reference herein.

For multivalent antigen binding proteins according to the invention, the covalent or noncovalent association of two or more SCA polypeptides is preferred for their formation. Although, multivalent SCAs can be produced from SCA with linkers as long as 25 residues, they tend to be unstable. Holliger, P., et al., Proc. Natl. Acad. Sci. (USA) 90:6444-6448 (1993), have recently demonstrated that linkers 0 to 15 residues in length facilitate the formation of divalent Fvs. See, Whitlow, M., et al., Protein Engng 7:1017-1026 (1994); Hoogenboom, H. R., Nature Biotech. 15:125-126 (1997); and WO 93/11161, incorporated by reference herein.

Identification and Synthesis of Site-specific PEGylation Sequences

The invention provides for thiol functional moieties, e.g., a thiol-containing amino acid residue located at specific sites in the V_{L} and V_{H} regions, adjacent to the C-terminus of the polypeptide (V_{L}, V_{H} or a neighboring site thereof), the N-terminus of the polypeptide (V_{L}, V_{H} or neighboring site thereof), the linker region between the first and second polypeptide regions, or in a combination of these regions. In the present invention, it is preferred that specific sites for polymer conjugation be located in the polypeptide linker, the C-terminus or adjacent to the C-terminus of the SCA, and preferably, at the second residue of the linker.

The thiol-containing functional group can be any known to art, including natural amino acid residues, and/or non-naturally-occurring amino acid residues, as well has thiol-functionalized derivatives of the same. In a preferred embodiment, the thiol functional moiety is a cysteine residue. This is preferred because SCA proteins normally have two buried disulfide bonds (Padlan E A, 1994, Antibody-Antigen Complexes, R. G. Landes Company, Austin), but no free cysteines. Thus, only the engineered Cys thiols are available for conjugation with activated polymers selective for reaction with thiols.

The particular nucleotide sequence which is used to introduce a Cys site into the various positions will depend upon the naturally-occurring nucleotide sequence. The most preferred sites are those in which it takes a minimum number of changes to create the Cys insertion while meeting the above-described steric requirements, as well. Of course, based on the redundancy of the genetic code, a particular amino acid may be encoded by multiple nucleotide sequences.

Any suitable art-known method for site-directed mutagenesis is used to change the native protein sequence to one that incorporates the Cys residue. The mutant protein gene is placed in an expression system, such as bacterial cells, yeast or other fungal cells, insect cells or mammalian cells. The mutant protein is then purified by standard methods for recovery of proteins.

Preferably, nucleic acid molecules expressing SCA muteins are produced by oligonucleotide-directed mutagenesis. Such methods for generating the site-specific Cys muteins, and related techniques for mutagenesis of cloned DNA, are well known in the art. See, Sambrook et al., MOLECULAR CLONING: A LABORATORY MANUAL, 2nd ed., Cold Spring Harbor Laboratory, Cold Spring Harbor, N.Y. (1989); Ausubel et al. (eds.), CURRENT PROTOCOLS IN MOLECULAR BIOLOGY, John Wiley and Sons (1987), both incorporated herein by reference.

Hosts and Vectors

After mutating the nucleotide sequence of an SCA of interest to provide for a Cys residue at the desired location, the mutated nucleic acid is preferably inserted into a suitable cloning vector, where the nucleotide encoding the SCA is operably linked to regulatory sequences controlling transcriptional expression. These are preferably selected by art-known techniques in order to optimize SCA production from a desired host cell system.

SCAs are known to be expressed and produced by prokaryotic or eukaryotic host cells, although for many purposes eukaryotic host cells are preferred. Preferred prokaryotic hosts include, but are not limited to, bacteria such as Bacillus, Streptomyces, Streptococci, and/or Escherichia coli . Preferred eukaryotic host cells include yeast or other fungal cells, insect cells and/or mammalian cells. Preferably, these include human or primate cells, present either in vivo, or in tissue culture. More preferably, the inventive SCAs are produced by transformed yeast, such as Pichia pastoris. Expression vectors are optionally selected to provide transient expression in a host cell, or to integrate into the host cell genome to create a transformed cell line.

Standard protein purification methods are used to purify these mutant proteins. Only minor modification to the native protein purification scheme may be required. For example, selection of vectors, hosts, methods of production, isolation and purification of monovalent, multivalent and fusion forms of proteins, especially SCA polypeptides, are thoroughly described by e.g., co-owned U.S. Pat. Nos. 4,946,778 and 6,323,322 incorporated by reference herein.

In one preferred embodiment, a nucleic acid molecule encoding an SCA of interest and a selection marker is integrated into a host cell chromosome, either as a single vector or as co-introduced vectors. The marker may complement an auxotrophy in the host (such as his_{4,} leu2, or ura3, which are common yeast auxotrophic markers), biocide resistance, e.g., antibiotics, or resistance to heavy metals, such as copper, or the like. The selectable marker gene can either be directly linked to the SCA DNA sequence to be expressed, or introduced into the same cell by co-transfection. Cells which have stably integrated the introduced nucleic acid are selected by survival or other effects of the marker in a given system.

In another embodiment, the SCA of interest is encoded by a suitable plasmid vector capable of autonomous replication in the recipient host cell. Any of a wide variety of art-known vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Any of a series of yeast vector systems can be utilized. Examples of such expression vectors include the yeast 2-micron circle, the expression plasmids YEP13, YCP and YRP, etc., or their derivatives. Such plasmids are well known in the art (Botstein et al., Miami Wntr. Symp. 19:265-274 (1982); Broach, J. R., In: The Molecular Biology of the Yeast Saccharomyces: Life Cycle and Inheritance, Cold Spring Harbor Laboratory, Cold Spring Harbor, N.Y., p. 445-470 (1981); Broach, J. R., Cell 28:203-204 (1982)). 115

For a mammalian host, several art-known vector systems are available. One class of vectors utilize DNA elements which provide autonomously replicating extra-chromosomal plasmids, derived from animal viruses such as bovine papilloma virus, polyoma virus, adenovirus, or SV40 virus. A second class of vectors relies upon the integration of the desired gene sequences into the host chromosome. Cells which have stably integrated the introduced DNA into their chromosomes are marker selected as discussed supra. Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol. 3:280 (1983), and others well known to the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Preferred vectors for expression in Pichia are pHIL-S 1 (Invitrogen Corp.) and pPIC9 (Invitrogen Corp.). Other suitable vectors will be readily apparent to the skilled artisan.

Once the vector or DNA sequence containing the constructs has been prepared for expression, the DNA constructs may be introduced or transformed into an appropriate host. Various techniques may be employed, such as transformation, transfection, protoplast fusion, calcium phosphate precipitation, electroporation, or other conventional techniques. After the cells have been transformed with the recombinant DNA (or RNA) molecule, the cells are grown in media and screened for appropriate activities. Expression of the sequence results in the production of the mutant SCA for PEG conjugation of the present invention.

Production and Purification of SCA Proteins

The monovalent or multivalent antigen-binding proteins of the invention can be produced by any suitable art-known methods. Broadly, the method includes preparing a suitable expression vector, expressing the vector in a compatible host cell, culturing the host cells and recovering the desired protein.

For expression in prokaryiotic cells or other cultured cells not capable of secreting the recombinant protein into the culture medium, the recovery is from the harvested cells. The harvested cellular material is subjected to cell lysis and washing, solubilization of the formed inclusion bodies in a compatible denaturing solvent, refolding by dilution under conditions effective to provide refolding into a function binding protein, and two ion-exchange HPLC chromatography steps. Preferred prokaryotic expression systems include, e.g., Escherichia coli (“E. coli”) See, for example, U.S. Pat. Nos. 4,946,778, 5,260,203, 5,455,030, 5,518,889, AND 5,534,621, as well as Bird et al., Science 242:423 (1988), also incorporated by reference herein.

Initial work on expression of the exemplified SCA proteins employed the E. coli expression system obtained from Xoma Corporation (araB promoter and pelB signal). The SCA protein was successfully expressed. However, the proteins expressed by the Xoma Corp. system remained cell associated in the periplasm, and would have required additional purification steps.

A more preferred expression system employs eukaryotic host cells and an expression vector with a secretion signal sequence. This preferred embodiment avoids the need to recover the SCA expressed as insoluble inclusion bodies from E. coli host cells. glycosylation, where needed. A number of recombinant DNA strategies exist which utilize strong promoter sequences and high copy number of plasmids which can be utilized for production of the desired proteins in yeast. Yeast recognizes leader sequences on cloned mammalian gene products, and secretes peptides bearing leader sequences (i.e., pre-peptides).

For this reason, the SCA proteins exemplified herein were all expressed by secretion from the yeast Pichia pastoris and recovered from solution.

SCA Proteins with D2E7 MAb Variable Domains

In a particularly preferred embodiment, SCAs according to the invention were developed employing variable domains of the D2E7 MAb. The D2E7 MAb was developed by Cambridge Antibody Technology and BASF Corporation. It binds specifically to a human cytokine, tumor necrosis factor alpha (TNF-α), and the MAb is described in detail by U.S. Pat. Nos. 6,09,0382, 6,258,562, incorporated by reference herein.. Selected domains of this antibody served as models to prepare a number of exemplary SCA molecules having one or more Cys residues incorporated into their respective polypeptide sequences at specific locations.

In brief, a wholly synthetic gene was constructed by polymerase chain reaction (PCR) using 14 long overlapping synthetic oligonucleotides, ranging from 20 bases to 102 bases in length,. Oligonucleotide-directed mutagenesis was further employed to construct other variants of the original sequence. The SCAs encoded by the resulting vectors contain the complete variable light (V_{L}) and variable heavy (V_{H}) segments of the D2E7 mAb, connected by a peptide linker. The exemplified linkers were an eighteen residue linker designated as the “218-linker,” and a 15 residue linker.

The eighteen amino acid “218-linker” has been described by Filpula et al, 1996, Antibody Engineering: A Practical Approach, 1996, Oxford University Press, pp 253-268). The 15 amino acid long (GGGGS)₃ linker has been described by Huston J S et al, 1988, Proc Natl Acad Sci (USA) 85: 5879-5883. In some cases, a six-histidine tag (his₆) at the C-terminus was included to simplify purification via metal immobilized metal ion-affinity chromatography (IMAC). The completed genes were cloned into an E. coli plasmid for DNA sequence confirmation on an ABI PRISM® 310 Genetic Analyzer from Applied Biosystems (Foster City, Calif.) (formerly produced by ABI/Perkin-Elmer). The domain orientations, linkers, and placement of the free cysteine are summarized in Table 1, below.

TABLE 1
SCA CLONES WITH D2E7 Fv

| SCA Clone | | | Free Cys at | |
|---|---|---|---|---|
| Nos. | SEQ ID NOs | DESIGN | Position(s) | FIGS. |
| 2-7-SC-1 | SEQ ID NO:1 | V_{L}-218-V_{H}-his₆ | None | 1A |
| 2-7-SC-2 | SEQ ID NO:2 | V_{L}-218-V_{H}-his₆ | C-terminus | 1B |
| 2-7-SC-3 | SEQ ID NO:3 | V_{H}-(GGGGS)₃- | C-terminus | 1C |
| | | V_{L}-his₆ | | |
| 2-7-SC-4 | SEQ ID NO:4 | V_{L}-218-V_{H} | C-terminus | 1D |
| 2-7-SC-5 | SEQ ID NO:5 | V_{L}-218-V_{H}-his₆ | Linker posi- | 1E |
| | | | tion 2 | |
| 2-7-SC-6 | SEQ ID NO:6 | V_{L}-218-V_{H}-his₆ | Linker posi- | 1F |
| | | | tion 2 and C- | |
| | | | terminus | |
| 2-7-SC-7 | SEQ ID NO:7 | V_{H}-(GGGGS)₃- | Linker posi- | 1G |
| | | V_{L}-his₆ | tion 2 | |
| 2-7-SC-8 | SEQ ID NO:8 | V_{L}-218-V_{H}-his₆ | Both N- and | 1H |
| | | | C-terminus | |
| 2-7-SC-9 | SEQ ID NO:9 | V_{L}-GGGGS-V_{H}- | None | 1-I |
| | | his₆ | | |

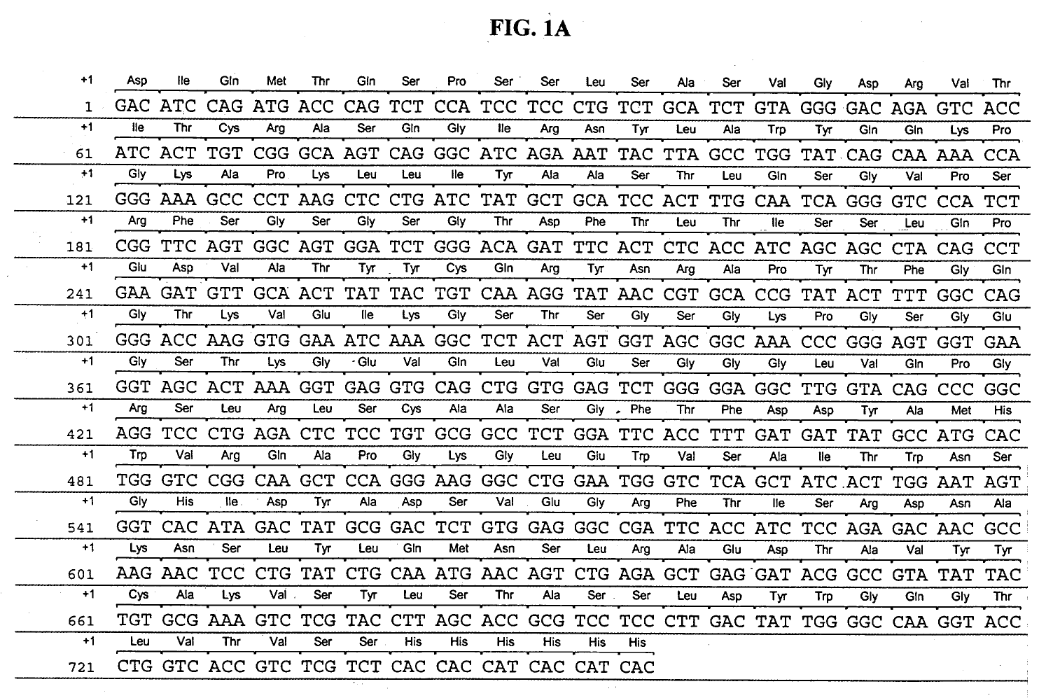

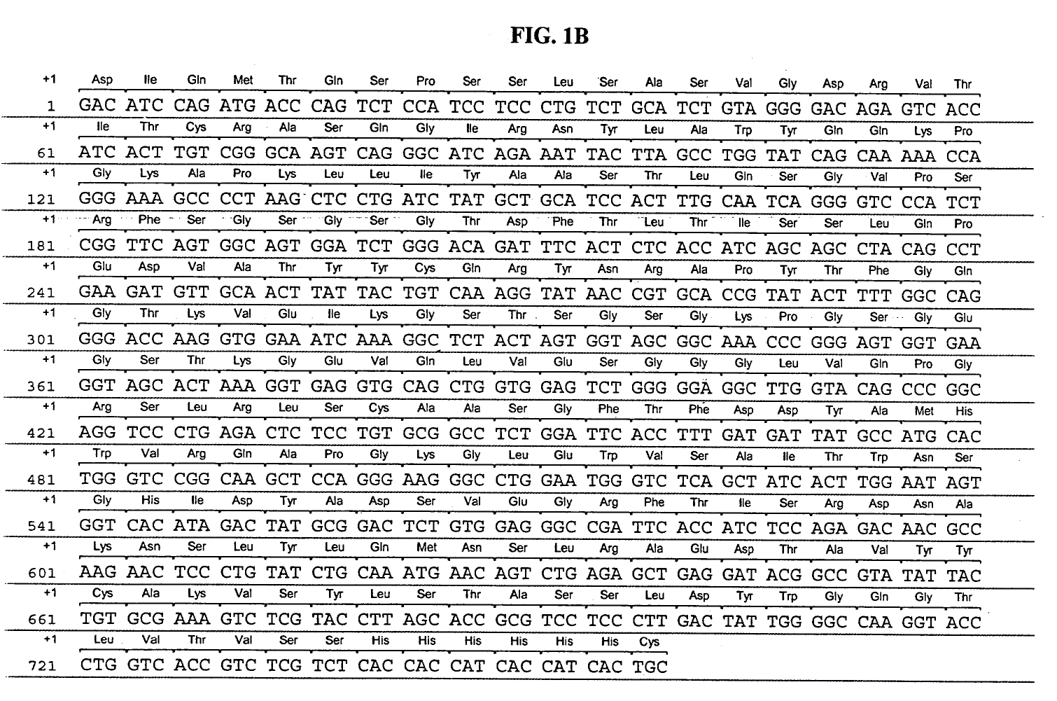

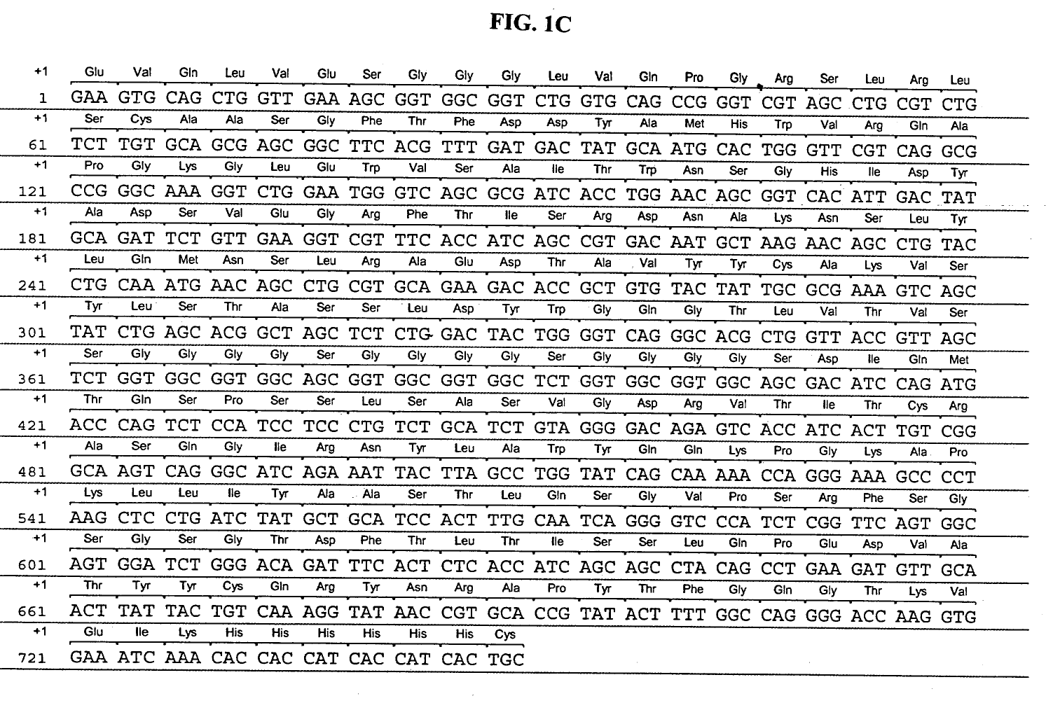

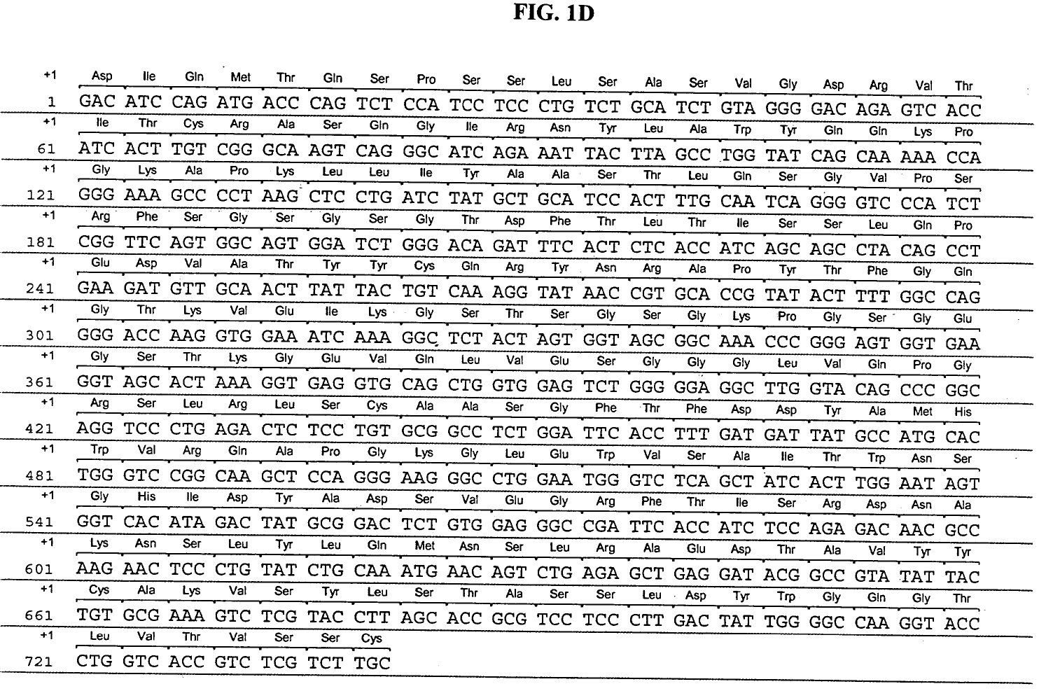

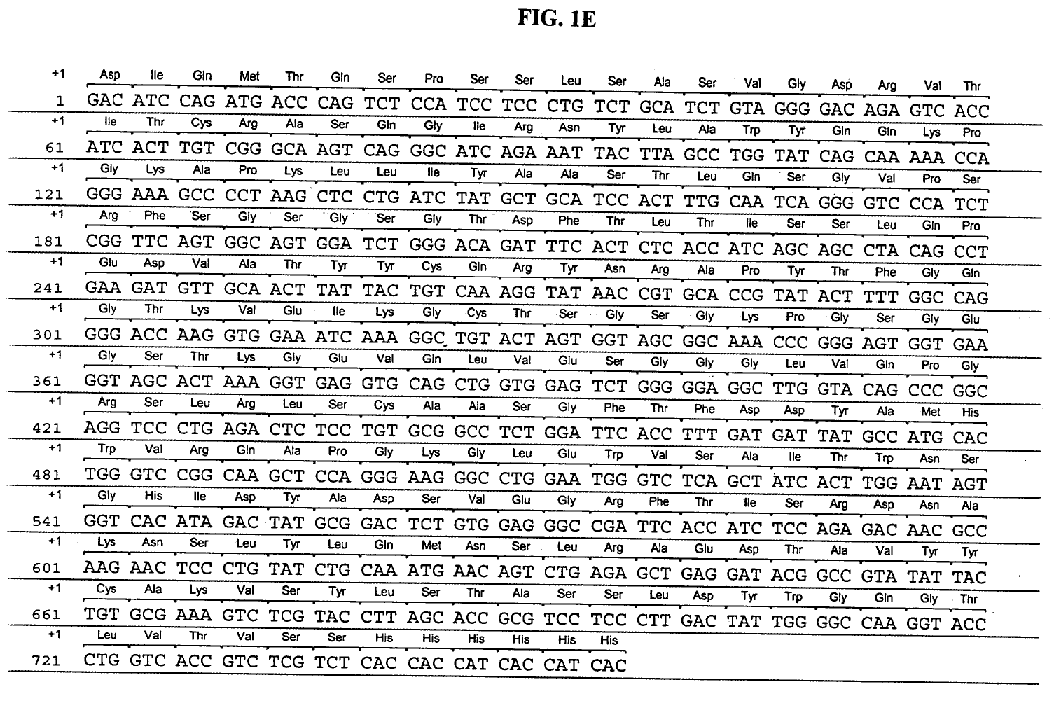

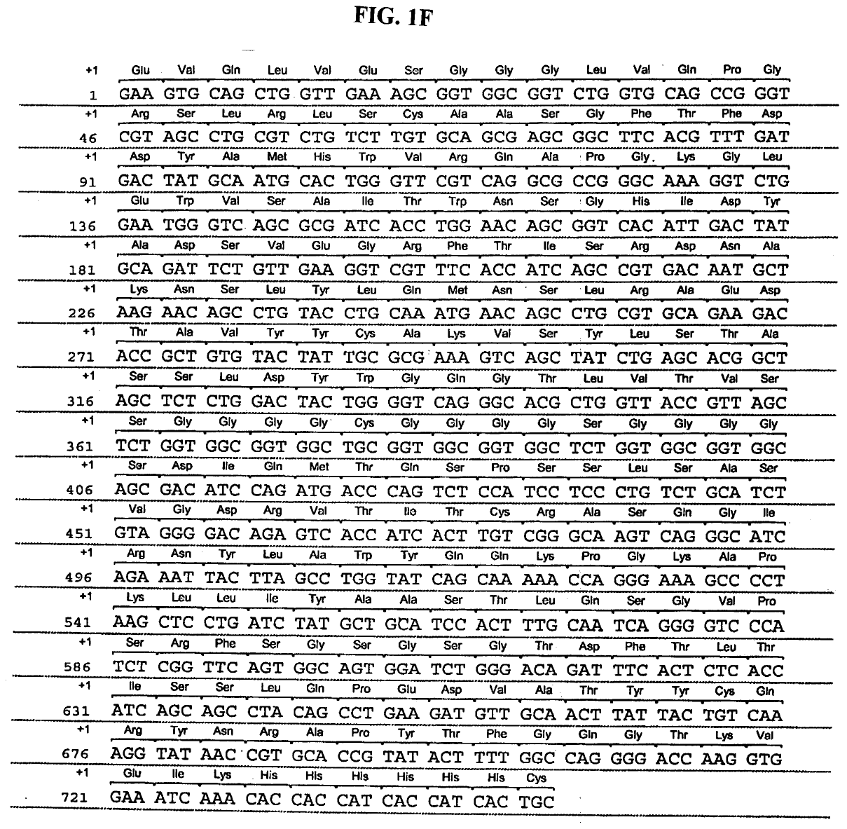

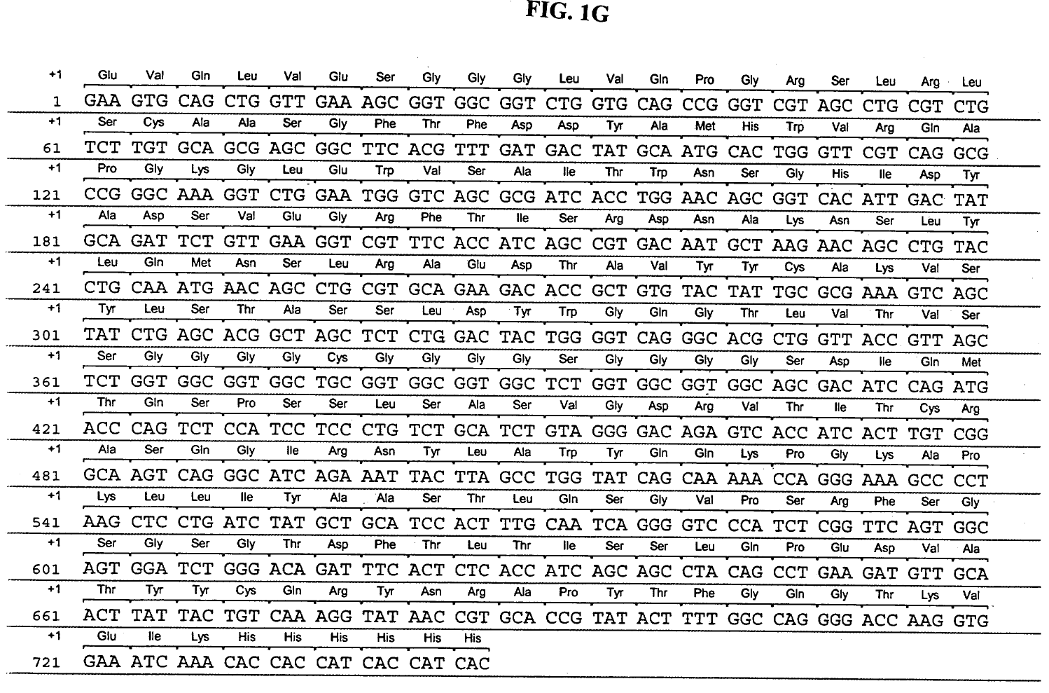

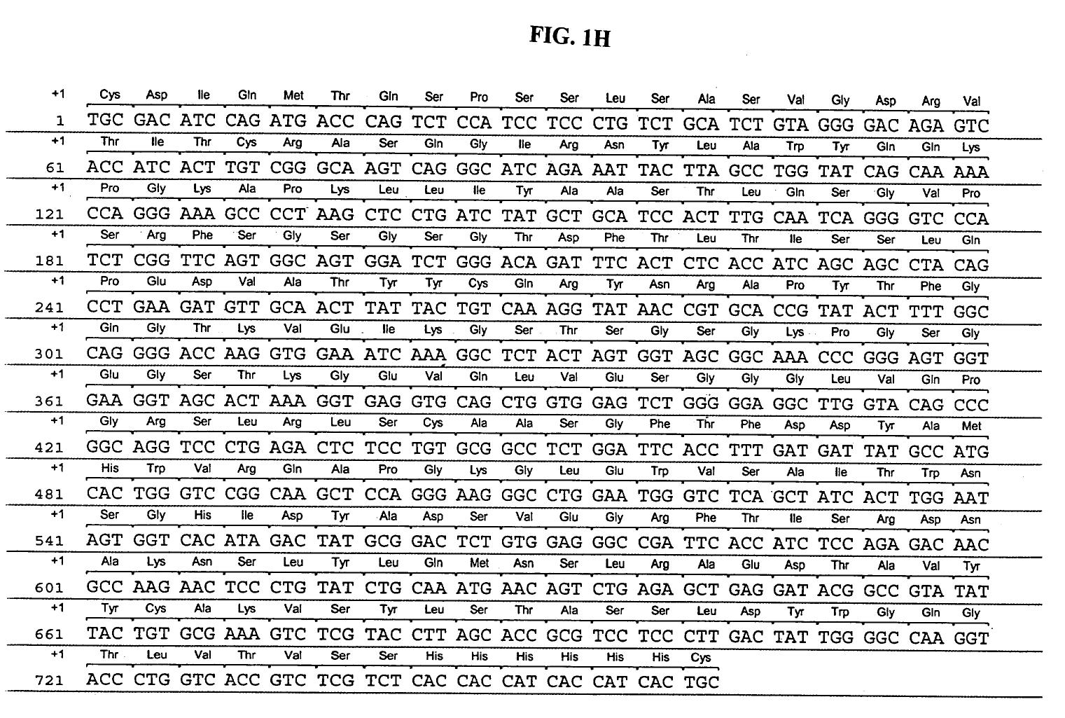

As summarized by Table 1, above, FIGS. 1A, 1B, 1C, 1D, 1E, 1F, 1G, 1H and 1-I present the DNA and encoded polypeptide sequences of the above nine genes.

Recombinant Expression and Purification of SCA Proteins.

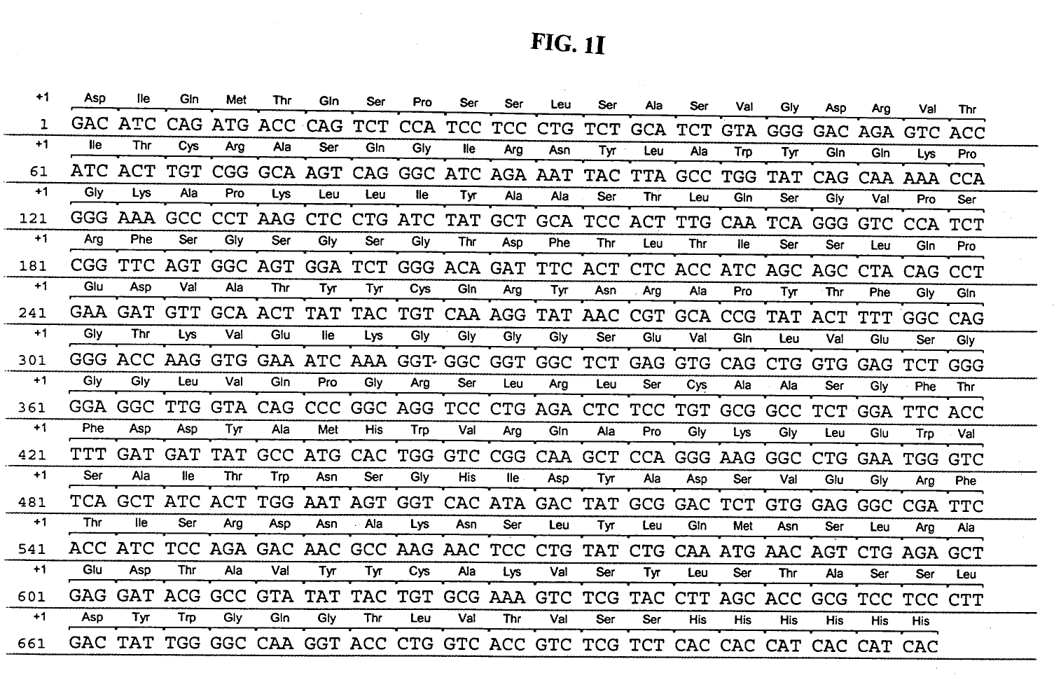

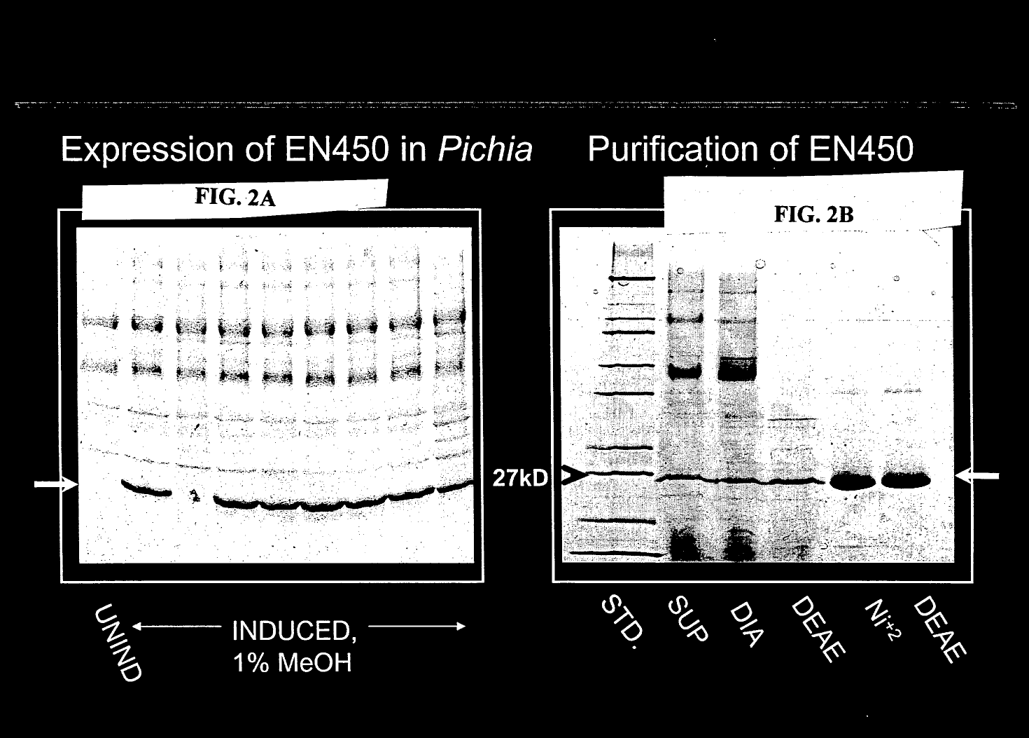

As noted above, Pichia pastoris was employed for production of the SCA variant proteins described above. The signal sequence from the yeast alpha mating factor was inserted directly in front of the mature coding sequence for each of these SCA proteins. The amino acid sequence of this signal peptide is Met Arg Phe Pro Ser Ile Phe Thr Ala Val Leu Phe Ala Ala Ser Ser Ala Leu Ala \hat{ } Ala (SEQ ID NO: 10) where the “\hat{ }” indicates the cleavage site. A 20^{th} amino acid of the signal (the alanine after the \hat{ }) was also included in these constructs. Therefore, the amino terminus of the mature SCA protein contains this alanine, followed, in each exemplified SCA, by the complete amino acid sequences illustrated by FIGS. 2A-2H (SEQ ID NOS. 1-9), respectively; as enumerated by Table 1). N-terminal protein sequence analysis confirmed that these sequences were correctly processed. The mutant genes expressing the D2E7 SCAs were individually ligated at the EcoRI site into the Pichia transfer plasmid pHIL-D2 (Invitrogen Corp.) and transformed into the yeast Pichia pastoris host GS-115.

Detailed protocols for these procedures are presented in the Pichia Expression Kit Instruction Manual Cat. No. X1710-01 (1994) from Invitrogen Corporation, incorporated by reference herein. Initial evaluation of expression was done by Coomassie Blue staining of SDS-PAGE gels. The clone numbers for the Pichia expression strains for each SCA protein correspond to the 2-7-SC numbers in Table 1, supra.

The SCA proteins (˜27 kDa) were expressed and secreted at high levels in recombinant Pichia (about 20-100 mg/L). Analysis on SDS-PAGE gels in the absence of reductant demonstrated the expected presence of both monomers and dimers formed by a single disulfide cross-link of two monomers. Rabbit antiserum versus the purified 2-7-SC-1 SCA protein was prepared. Western analysis with this reagent verified the identity of the expressed SCA proteins.

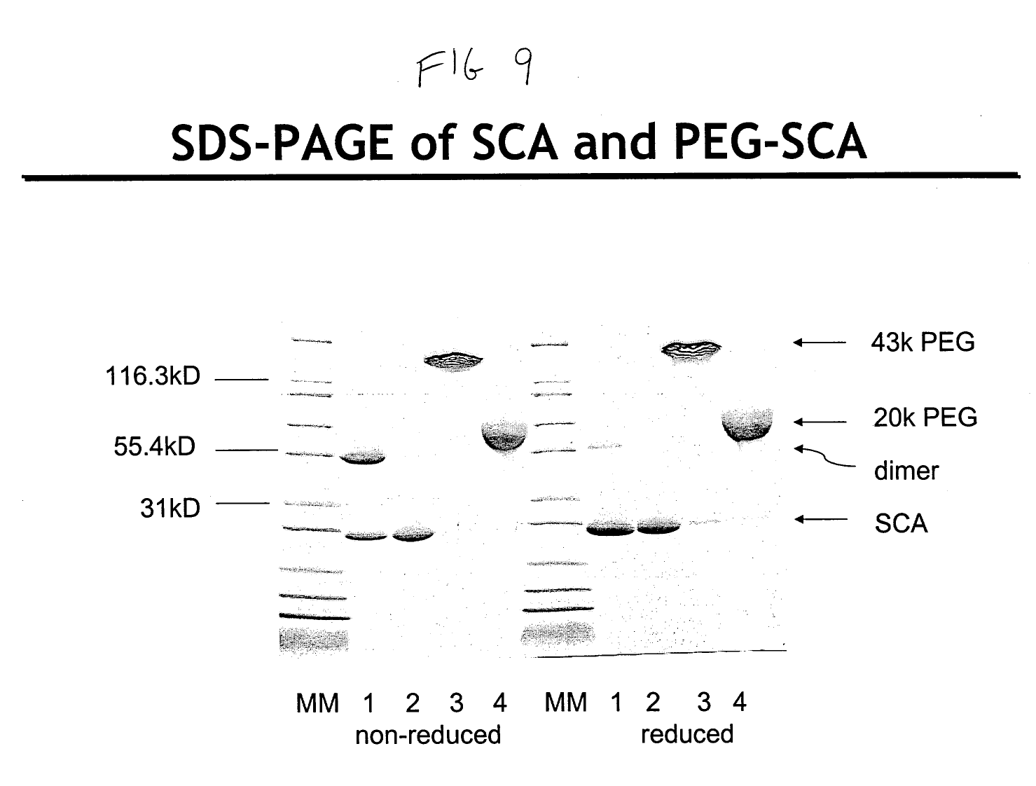

For example, FIG. 8 shows a representative Western blot analysis of D2E7 2-7-SC-2 and PEGylated forms confirming reactivity of this anti-D2E7 antiserum with the recombinant SCA proteins and PEG-SCA conjugates. Anti-218-linker antibody was employed. 1 μg of each samle was analyszed on 4-20% non-reducing SDS-PAGE gel. The primary and secondary antibodies are anti 18-linker antibody raised in rabbit and goat anti rabbit antibody conjugatd horserasish peroxidase, respectively. The enzyme substrate was TMBM peroxidase substrate from Moss, In. D2E7 2-7-SC-2 has PEG at c-terminal and 2-7-SC-5 has PEG at 218 linker. Band A is 2-7-SC-2, Band B is 2-7-SC-5, Band C is PEG(20k)-2-7-SC-2, Band D is PEG(20k)-2-7-SC-5, Band E is PEG(40k)-2-7-SC-2, Band F is PEG(40k)-2-7-SC-5.

The SCA proteins were purified from Pichia pastoris supernatants to greater than 95% purity by simple two or three column chromatography protocols. For the SCA proteins bearing a his₆ tag, the dialyzed fermentation medium was diafiltered and passed through a DEAE column, then bound to an IMAC nickel affinity column, (QIAGEN). The bound SCA protein was eluted with imidazole; then flowed through a second DEAE anion exchange column. The flow-through was diafiltered for concentration of the SCA protein and further characterized. For SCA proteins lacking the his₆ tag, the SCA protein was either purified on a protein L-agarose column with low pH elution, obtained from Pierce Biotechnology, Inc (Rockford, Ill.), or captured on HS cation exchange chromatography, obtained from Amersham Pharmacia (Piscataway, N.J.), followed by salt gradient elution and diafiltration. HS chromatography was the preferred method.

FIGS. 2A and 2B show representative expression and purification data for clone 2-7-SC-2, including the SDS-PAGE gel analysis by Coomassie Blue staining of the fractions, and the yield at each step. A small amount of ˜54 kDa disulfide-linked dimer is visible in the stained gel.

Thiol-Specific Activated Polymers

Preferably, the inventive SCAs are linked to thiol-specific activated polymers. Specifically, the activated polymers preferably employed in the present invention are those which have a sulfhydryl- or thiol-selective terminal linking group on at least one terminal thereof. Several art-known activated polymers, e.g., polyalkylene oxide (PAO) polymers that are reactive with free thiols, are readily employed in the practice of the invention.

Examples of reactive groups include maleimide, vinylsulfone, thiol, orthopyridyl disulfide, and iodoactemide, with maleimide activated polyethylene glycols (PEG-mal's) being more preferred, in view of the maleimide group being highly specific for thiols and the conjugation reaction taking place under mild conditions. See also, for example, co-owned U.S. Pat. No. 5,730,990, incorporated by reference herein. Additional sulfhydral selective activated PEG-polymers are also available from Nektar Therapeutics (formerly Shearwater Corporation) as illustrated by the 2001 Shearwater Corporation Catalog, incorporated by reference herein. See also Goodson, R. J. & Katre, N. V. 1990, Bio/Technology 8:343; Kogan, T. P. 1992, Synthetic Comm. 22, 2417, incorporated by reference herein.

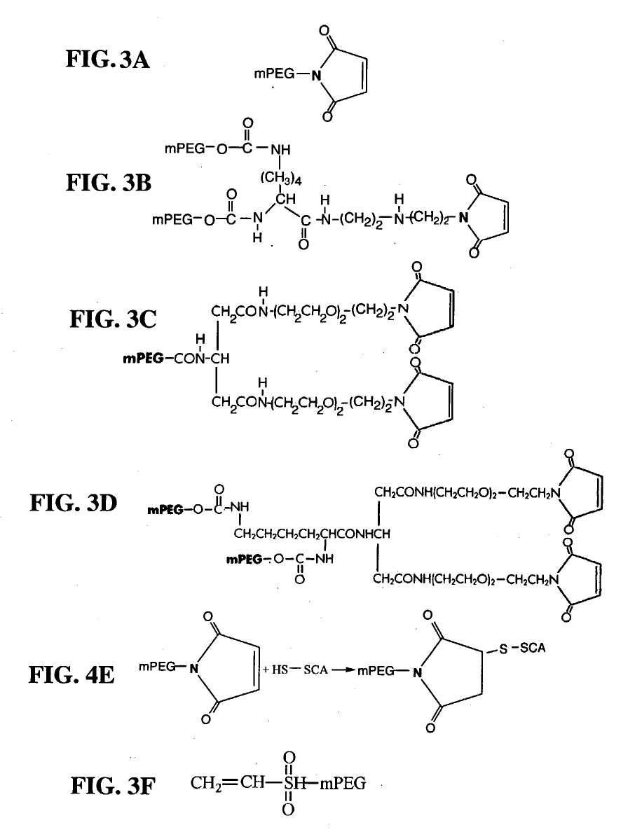

For example, the linear polymers mPEG-MAL (5 kDa) (e.g., Shearwater Cat. No. 2D3X0T01) and mPEG-MAL (20 kDa), as well as branched polymer mPEG2-MAL (40 kDa) (e.g., Shearwater Cat. No. 2D2M0H01) conjugated to inventive SCAs, are exemplified herein. Structures of these maleimide-PEG polymers and the conjugation chemistry are shown for convenience by FIGS. 3A-3E. FIG. 3F illustrates mPEG-vinylsulfone. The various maleimide-activated polymers readily react with free thiols, as illustrated by FIG. 3E. In FIG. 3E, “SCA” is a protein according to the invention having at least one free thiol (S-H). See Table 2, below.

TABLE 2

| | | Nektar/Shearwater |
|---|---|---|
| FIG. Nos. | Description | Cat. Nos. |
| | mPEG-MAL | 2D2M0H01, |
| | | 2D2M0P01 |
| | mPEG₂(MAL) | 2D3X0T01 |
| | mPEG(MAL)₂ | 2D2D0H0F, |
| | | 2D2D0P0F |
| | mPEG₂(MAL)₂ | Catalog, page 10 |
| | mPEG Vinylsulfone | |

Additional polymeric platforms which can include the sulfhydryl-specific linkers include those disclosed in commonly-assigned U.S. Pat. Nos. 5,643,575, 5,919,455, 6,113,906, (U-PEG's), 6,153,655 and 6,395,266 (terminally branched PEG's), 6,251,382 (polyPEG's) and U.S. Ser. No. 10/218,167 (bicines), etc. See also Shearwater Polymers, Inc. catalog “Polyethylene Glycol and Derivatives 2001”. The disclosure of each of the foregoing is incorporated herein by reference.

As mentioned above, the polymer portion of the conjugate is preferably a polyalkylene oxide. More preferably, the polymer portion is a polyethylene glycol which is substantially non-antigenic. Although PAO's and PEG's can vary substantially in weight average molecular weight, those included in the compositions of the present invention independently have a weight average molecular weight of from about 2,000 Da to about 136,000 Da in most aspects of the invention. More preferably, the polymer has a weight average molecular weight of from about 3,000 Da to about 100,000 Da. Most preferably, the polymer portion has a weight average molecular weight of from about 5,000 Da to about 40,000 Da.

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Confirmation of the specific reactivity of the maleimide polymers with free cysteine, but not with lysine or histidine, was accomplished by reaction of the maleimides with these respective free amino acids.

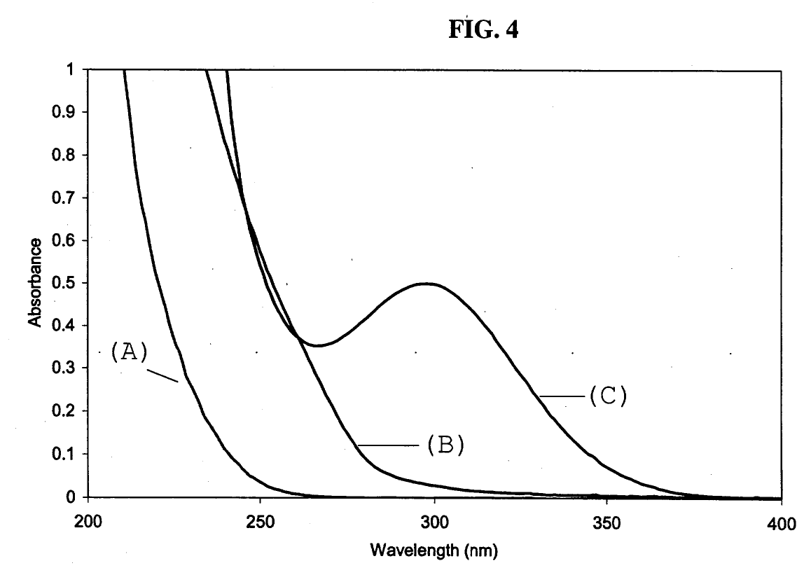

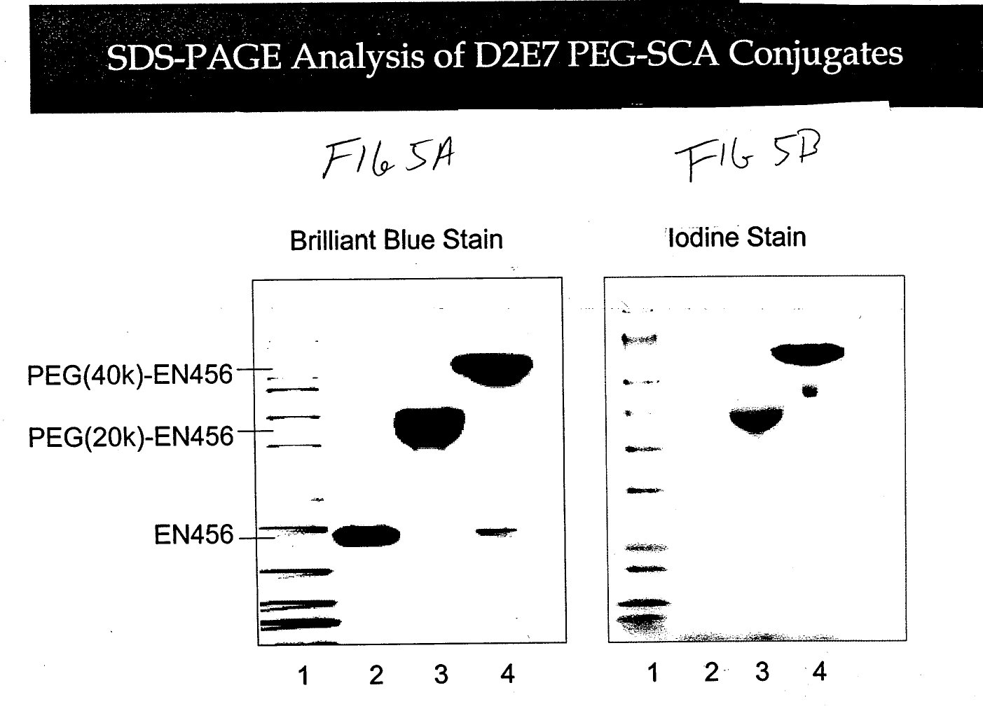

FIG. 4 confirms the reactivity of cysteine with activated PEG-MAL. The absorbance for cysteine, as a 3 mM solution, is shown as curve A. The absorbance curve for activated PEG-MAL, at a concentration of 1 mM, is shown as curve C and is characterized by a wide absorbance peak centered on 300 nm. Absorbance curve B was taken of a solution combining cysteine and activated PEG-MAL in a 1:3 ratio (1 mM PEG-MAL and 3 mM cysteine, 100 mM sodium phosphate, pH 6.0, 1 mM EDTA, 25° C.). The B curve tracks the

A curve, with a shift to the right, but the characteristic 300 nM broad peak of PEG-MAL, is not present, confirming the reactivity of cysteine with activated PEG-MAL. Analogous absorbance curves for histidine or lysine (not shown), confirm that these residues are not highly reactive under the employed conditions.

Labeled or Tagged Conjugates

Upon production of a polyalkylene oxide conjugated SCAs according to the invention, the conjugates are optionally further modified by linking or conjugating a diagnostic or therapeutic agent to the SCA-polymer conjugate. The general method of preparing an antibody conjugate according to the invention is described in Shih, L. B., et al., Cancer Res. 51:4192 (1991); Shih, L. B., and D. M. Goldenberg, Cancer Immunol. Immunother. 31:197 (1990); Shih, L. B., et al., Intl. J. Cancer 46:1101 (1990); Shih, L. B., et al., Intl. J. Cancer 41:832 (1988), all incorporated herein by reference. The indirect method involves reacting an antibody (or SCA), whose polyalkylene oxide has a functional group, with a carrier polymer loaded with one or plurality of bioactive molecules, such as, peptides, lipids, nucleic acids (i.e., phosphate-lysine complexes), drug, toxin, chelator, boron addend or detectable label molecule(s).

In certain alternative embodiments, the polyalkylene oxide conjugated SCA is directly conjugated or linked to a diagnostic or therapeutic agent. The general procedure is analogous to the indirect method of conjugation except that a diagnostic or therapeutic agent is directly attached to an oxidized sFv component. See Hansen et al., U.S. Pat. No. 5,443,953, incorporated herein by reference.

Pharmaceutical Compositions and Administering the SCA and/or SCA-Polymer Conjugates

The pharmaceutical compositions of the invention may include a “therapeutically effective amount” or a “prophylactically effective amount” of an SCA and/or SCA-polymer conjugate of the invention. A “therapeutically effective amount” refers to an amount effective, at dosages and for periods of time necessary, to achieve the desired therapeutic result. A therapeutically effective amount of the SCA and/or SCA-polymer conjugate may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an SCA and/or SCA-polymer conjugate of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Pharmaceutical Compositions

In a further preferred embodiment, the SCAs of the invention are employed for treating and/or diagnosing conditions related to the binding specificity of a particular SCA protein of interest. Thus, an SCA and/or SCA-polymer conjugate is administered by art-known methods, to an animal or person having a disease or disorder for which the binding properties of the administered SCA are useful in treating or diagnosing such disease or disorder. Preferably, the SCA is polymer-conjugated according to the invention.

The SCAs and conjugated SCAs of the invention can be incorporated into pharmaceutical compositions suitable for administration to a subject, e.g., an animal or person in need of such administration. Typically, the pharmaceutical composition comprises an SCA polypeptide having at least one type of binding specificity, and a pharmaceutically acceptable carrier.

The term, “pharmaceutically acceptable carrier” includes any and all solvents, dispersion media, coatings, antimicorbial, e.g., antibacterial and antifungal agents, isotonic and absorption delaying agents, and the like, that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition.

Pharmaceutically acceptable carriers may further comprise minor amounts of auxiliary substances such as wetting or emulsifying agents, preservatives or buffers, which enhance the shelf life or effectiveness of the antibody or antibody portion.

The inventive compositions are optionally prepared in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular).

In a preferred embodiment, the SCA and/or SCA-polymer conjugate is administered by intravenous infusion or injection. In another preferred embodiment, the antibody is administered by intramuscular or subcutaneous injection. Administration via inhalation, as a spray, aerosol or mist is also contemplated where that route is advantageous, e.g., for systemic absorption and/or local action within the respiratory system.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The SCAs and/or SCA-polymer conjugates of the present invention can be administered by a variety of methods known in the art, although for many therapeutic applications, the preferred route/mode of administration is intravenous injection or infusion. As will be appreciated by the skilled artisan, the route and/or mode of administration will vary depending upon the desired results.

In certain embodiments, the SCAs and/or SCA-polymer conjugates of the invention may be orally administered, for example, with an inert diluent or an assimilable edible carrier. The SCAs and/or SCA-polymer conjugates (and other ingredients, if desired) are optionally enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer the SCAs and/or SCA-polymer conjugates of the invention by other than parenteral administration, it may be necessary to coat the compound with, or co-administer the compound with, a material to prevent its inactivation.

Supplementary active compounds can also be incorporated into the pharmaceutical compositions. In certain embodiments, an SCAs and/or SCA-polymer conjugate of the invention is co-formulated with and/or co-administered with one or more additional therapeutic agents that will provide additive, synergistic or supplementary therapeutic or diagnostic activity for a disease or disorder.

Administering Anti-hTNF-α SCAs and/or SCA-Polymer Conjugates

For example, anti-hTNFα SCAs and/or SCA-polymer conjugates of the invention may be co-formulated and/or co-administered with one or more additional antibodies or SCAs that bind other targets (e.g., that bind other cytokines or that bind cell surface molecules), one or more cytokines, soluble hTNFα receptors (see e.g., PCT Publication No. WO 94/06476) and/or one or more chemical agents that inhibit hTNF-α production or activity (such as cyclohexane-ylidene derivatives as described in PCT Publication No. WO 93119751). Furthermore, one or more SCAs and/or SCA-polymer conjugates of the invention may be used in combination with two or more of the foregoing therapeutic agents. Such combination therapies may advantageously utilize lower dosages of the individual administered therapeutic agents.

Indications for Anti-TNFα -SCAs and Preferred Co-Administered Agents

Simply by way of a example, U.S. Pat. Nos. 6,258,562 and 6,090,382, incorporated by reference herein, provide an exhaustive list of diseases and disorders for which TNFα is a mediator or co-mediator of primary or other aspects of disease processes. Art-known agents for treating or palliating such diseases or disorders are contemplated to be co-formulated or co-administered with the anti-TNFα embodiments of the inventive SCAs and/or polymer conjugated SCAs. In brief, the list of diseases and disorders mediated by or related to the actions of TNFα, and therefore rationally treated by a TNFα binder, optionally in combination with other art-known therapeutics, is stated by U.S. Pat. No. 6,258,562 to include, e.g., sepsis, autoimmune diseases, infectious diseases, transplantation/rejection, malignancy, pulmonary and intestinal disorders.

These indications and, where applicable, agents that are optionally co-formulated in treating such indications with anti-TNFα SCAs, are as follows.

Sepsis.

Treatable conditions associated with sepsis include TNFα—mediated septic shock syndrome and associated hypotension, myocardial suppression, vascular leakage syndrome, organ necrosis, stimulation of the release of toxic secondary mediators and activation of the clotting cascade, endotoxic shock, gram negative sepsis and toxic shock syndrome.

Autoimmune Diseases.

Treatable conditions associated with auto immune diseases include, tissue inflammation and joint destruction in rheumatoid arthritis, death of islet cells and the insulin resistance in diabetes, cytotoxicity to oligodendrocytes and induction of inflammatory plaques in multiple sclerosis. Agents contemplated to be co-formulated or co-administered with the inventive anti-TNFα SCAs and/or polymer conjugated SCAs include any art-known agents available to treat such autoimmune disorders including, e.g., glucocorticosteroids, non-steroidal anti-inflammatory drug(s) (NSAIDs); cytokine suppressive anti-inflammatory drug(s) (CSAIDs); CDP-5711 BAY-10-3356 (humanized anti-TNFα antibody; Celltech/Bayer); cA2 (chimeric anti-TNFα antibody; Centocor); 75 kdTNFR-IgG (75 kD TNF receptor-IgG fusion protein; Immunex; see e.g., Arthritis & Rheumatism (1994) Vol. 37. S295; J. Invest Med. (1996) Vol. 44 235A); 55 kdTNFR-IgG (55 kD TNF receptor-IgG fusion protein; Hoffmann-LaRoche); IDEC-CE9.I/SB 210396 (non-depleting primatized anti-CD4 antibody; IDEC/SmithKline, to name but a few.

Infectious Diseases.

Treatable conditions associated with infectious diseases include TNFα—mediated brain inflammation, capillary thrombosis and infarction in malaria, venous infarction in meningitis, cachexia secondary to invention, e.g., HIV virus invention, stimulation of viral proliferation and central nervous system injury in HIV infection, fever and myalgias due to infections such as influenza). Agents contemplated to be co-formulated or co-administered with the inventive anti-TNFα SCAs and/or polymer conjugated SCAs include any art-known anti-infective agents, e.g., antibiotics, anti-bacterials, antivirals, and the like, as well as non-steroidal anti-inflammatory drug(s) (“NSAIDs”) and/or antibodies or SCAs that bind to the infective agent and/or its toxins or essential components.

Transplantation Treatable conditions associated with transplantion medicine, rejection of transplants or side effects of the required immunosuppression agents include TNFα—mediated allograft rejection and graft versus host disease (GVHD), to name but a few transplantation-related effects. Agents contemplated to be co-formulated or co-administered with the inventive anti-TNFα SCAs and/or polymer conjugated SCAs include, e.g., glucocorticosteriods, cyclosporin A, FK506, and/or OKT3, to inhibit OKT3-induced reactions, as well as in combination with binders directed to immune cell receptors such as antibodies or SCAs binding to CD25 (IL-2 receptor-α),CD11a (LFA-1), CD54 (ICAM-1), CD4, CD45, CD28/CTLA4, CD80 (B7-1) and/or CD86 (B7-2).

Malignancy. Treatable conditions associated with malignancy include TNFα—mediated cachexia, tumor growth, metastatic potential and cytotoxicity in malignancies. Agents contemplated to be co-formulated or co-administered with the inventive anti-TNFα SCAs and/or polymer conjugated SCAs include any art-known anti-tumor or anti-cancer agents.

Pulmonary Disorders,

Treatable conditions associated with pulmonary disorders include adult respiratory distress syndrome, shock lung, chronic pulmonary inflammatory disease, pulmonary sarcoidosis, pulmonary fibrosis and silicosis. For pulmonary disorders, the inventive anti-TNFα SCAs and/or polymer conjugated SCAs are optionally administered by oral or nasal spray, or formulated for administration as an aerosol, via any standard inhalation system. Such formulations can be co-administered or administered at alternate times with other agents suitable for treating such pulmonary disease or disorder, or an agent that facilitates bronchial access for the SCA formulation.

Intestinal Disorders.

Treatable conditions associated with intestinal disorders include the range of inflammatory bowel disorders, e.g., Crohn's disease and/or ulcerative colitis. Agents contemplated to be co-formulated or co-administered with the inventive anti-TNFα SCAs and/or polymer conjugated SCAs include, budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β MAbs; anti-IL-6 MAbs; growth factors; elastase inhibitors; pyridinyl-imidazole compounds; CDP-571/BAY-10-3356 (humanized anti-TNFα antibody; Celltech/Bayer); cA2 (chimeric anti-TNFα antibody; Centocor); 75 kdTNFR-IgG (75 kD TNF receptorIgG fusion protein; Immunex; 55 kdTNFR-IgG (55 kD TNF receptor-IgG fusion protein; Hoffmann-LaRoche); interleukin-10 (SCH 52000; Schering Plough); IL4; IL-10 and/or IL4 agonists (e.g., agonist antibodies); interleukin-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of Platelet Activating Factor (PAF); ciprofloxacin; and lignocaine.

Diagnostic and Assay Methods: Anti-TNFα SCA or SCA-Conjugates

The anti-hTNFα SCAs and/or SCA-polymer conjugates of the invention can be used to detect hTNFα in samples of interest, such as in a biological sample, including serum or plasma or other clinical specimens, using a conventional immunoassay. These include enzyme linked immunosorbent assays (ELISA), radioimmunoassay (RIA) or tissue immunohistochemistry.

The invention provides a method for detecting hTNFα in a biological sample comprising contacting a biological sample with an antibody, or antibody portion, of the invention and detecting either the antibody (or antibody portion) bound to hTNFα or unbound SCA and/or SCA-polymer conjugates, to thereby detect hTNFα in the biological sample. The SCA is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

In an optional embodiment, the inventive SCAs are not labelled, but hTNFα is assayed in biological fluids by a competition immunoassay wherein rhTNFα standards are labeled with a detectable substance, and an unlabeled anti-hTNFα SCA and/or SCA-polymer conjugate. In this assay, the biological sample, the labeled rhTNFα standards and the anti-hTNFα SCA and/or SCA-polymer conjugate are combined and the amount of labeled rhTNFα standard bound to the unlabeled SCA and/or SCA-polymer conjugate is determined. The amount of hTNFα in the biological sample is inversely proportional to the amount of labeled rhTNF_ standard bound to the anti-hTNFα SCA and/or SCA-polymer conjugate.

U.S. Pat. Nos. 6,258,562 and 6,090,382 indicate that the D2E7 MAb can also be used to detect TNFα s from species other than humans, in particular TNFαs from primates (e.g., chimpanzee, baboon, marmoset, cynomolgus and rhesus), pig and mouse, it is contemplated that the anti-TNFα SCA and/or SCA-polymer conjugates of the invention are readily employed for that purpose, as well.

### EXAMPLES

The following examples serve to provide further appreciation of the invention but are not meant in any way to restrict the effective scope of the invention.

### Example 1

Design of SCA Proteins with at Least One Free Thiol.

Nine SCA polypeptides were designed based on the variable domains of the D2E7 MAb. The use of the term, “D2E7 SCA” herein refers to any of the SCA produced with the D2E7 variable domains as exemplified herein, unless otherwise indicated. Each was constructed as follows.

As noted supra a wholly synthetic gene was constructed by polymerase chain reaction (PCR) using 14 long overlapping synthetic oligonucleotides, ranging from 20 bases to 102 bases in length, ). Oligonucleotide-directed mutagenesis was further employed to construct other variants of the original sequence. The expressed SCA proteins contain the complete variable light (V_{L}) and variable heavy (V_{H}) segments of the D2E7 MAb, connected by a peptide linker. Two linkers were employed.

Linker “218” is an eighteen amino acid residue 218-linker described by Filpula et al., 1996 (Antibody Engineering: A Practical Approach, Oxford University Press, pp 253-268).

The “(GGGGS)₃ linker” is a 15 amino acid residue linker described by Huston J S et al, 1988, Proc Natl Acad Sci (USA) 85: 5879-5883. In some cases, as noted by Table 1, supra, a six-histidine tag (his₆) at the C-terminus was included to simplify purification via metal immobilized metal ion-affinity chromatography (“IMAC”).

The completed genes were cloned into an E. coli plasmid for DNA sequence confirmation on an ABI PRISM® 310 Genetic Analyzer from Applied Biosystems (Foster City, Calif.) (formerly produced by ABI/Perkin-Elmer). The domain orientations, linkers, and placement of the free cysteine in each respective SCA modeled on the D2E7 MAb, are summarized in Table 1, supra (clone numbers 2-7-SC-1 through -9).

The nucleic acid chains expressing each of clone numbers 2-7-SC-1 through -9 were prepared as follows.

Method of Cloning and Expression of D2E7 SCA

The synthetic V_{L}-V_{H} version of D2E7 SCA gene was constructed by two rounds of PCR using six overlapping oligonucleotides as templates for the V_{L} chain and six overlapping oligonucleotides as templates for V_{H} chain. The V_{L} chain and V_{H} chain of D2E7 SCA gene were linked with a 218 linker. The C-terminus of the encoded protein was followed by 6 tandem histidines for IMAC purification purposes.

Six oligonucleotides from 5′ to 3′ end for V_{L} were designed as follows:

| | | |
|---|---|---|
| V_{L}1: | GACATCCAGATGACCCAGTCTCCATCCTCCCTGTCTGCATCTGTAGGGGAC | (SEQ ID NO: 19) |
| V_{L}2: | GCATCTGTAGGGGACAGAGTCACCATCACTTGTCGGGCAAGTCAGGGCATCAGA | (SEQ ID NO: 20) |
| | AATTACTTAGCCTGGTATCAGCAAAAACCAGGGAAAGCCCCT | |
| V_{L}3: | CCCTGATTGCAAAGTGGATGCAGCATAGATCAGGAGCTTAGGGGCTTTCCCTGG | |
| V_{L}4: | TCCACTTTGCAATCAGGGGTCCCATCTCGGTTCAGTGGCAGTGGATCTGGGACA | (SEQ ID NO: 21) |
| | GATTTC | |
| V_{L}5: | TCTGGGACAGATTTCACTCTCACCATCAGCAGCCTACAGCCTGAAGATGTTGCA | (SEQ ID NO: 22) |
| | ACTTATTACTGTCAAAGGTATAACCGTGCACCGTATACTTTTGGCCAG | |
| V_{L}6: | ACCACTCCCGGGTTTGCCGCTACCACTAGTAGAGCCTTTGATTTCCACCTTGGT | (SEQ ID NO: 23) |
| | CCCCTGGCCAAAAGTATA. | |

Among them, V_{L}1, 2, 4 and 5 were forward (sense) oligonucleotides, and V_{L}3 and 6 were reverse oligonucleotides.

Six oligonucleotides from 5′ to 3′ end for V_{H} were designed as follows:

| | | |
|---|---|---|
| V_{H}1: | GGCAAACCCGGGAGTGGTGAAGGTAGCACTAAAGGTGAGGTGCAGCTGGTG | (SEQ ID NO: 24) |
| | GAGTCTGGGGGA | |
| V_{H}2: | GTGGAGTCTGGGGGAGGCTTGGTACAGCCCGGCAGGTCCCTGAGACTCTCC | (SEQ ID NO: 25) |
| | TGTGCGGCCTCTGGATTCACCTTTGATGATTATGCCATGCACTGGGTCCGG | |
| V_{H}3: | CCAAGTGATAGCTGAGACCCATTCCAGGCCCTTCCCTGGAGCTTGCCGGAC | (SEQ ID NO: 26) |
| | CCAGTGCAT | |
| V_{H}4: | TCAGCTATCACTTGGAATAGTGGTCACATAGACTATGCGGACTCTGTGGAG | (SEQ ID NO: 27) |
| | GGCCGATTC | |
| V_{H}5: | GTGGAGGGCCGATTCACCATCTCCAGAGACAACGCCAAGAACTCCCTGTAT | (SEQ ID NO: 28) |
| | CTGCAAATGAACAGTCTGAGAGCTGAGGATACGGCCGTATATTACTGTGCG | |
| V_{H}6: | AGACGAGACGGTGACCAGGGTACCTTGGCCCCAATAGTCAAGGGAGGACGC | (SEQ ID NO: 29) |
| | GGTGCTAAGGTACGAGACTTTCGCACAGTAATATAC | |

Among them, V_{H}1, 2, 4 and 5 were forward oligonucleotides and VH³ and 6 were reverse oligonucleotides. All oligonucleotide designed for the synthetic V_{L} and V_{H} of D2E7 SCA were synthesized by MWG Biotech, Inc.

The V_{L} of the D2E7 SCA gene was assembled in a first round PCR, using 2 mM Tris (pH8.4), 5 mM KCl, 7.5 mM MgCl₂,1.5 mM dNTP, 2 units of Platinum Tag polymerase (Invitrogen), oligonucleotides V_{L}1, 2, 3, 4, 5 and 6 (1 pmol each) as templates, 5′ TGGCGAGCTCTGACATCCAGATGACCCAGTCT (SEQ ID NO: 30) (50 pmol) as forward primer and 5′ACCACTCCCGGGTTTGCCGCTACCACTAGTAGA (SEQ ID NO: 31) (50 pmol) as reverse primer.

The PCR was performed for 30 cycles of 94° C. for 30 seconds, 56° C. for 30 seconds and 72° C. for 60 seconds, followed by 72° C. for 10 minutes.

The V_{H} of the D2E7 SCA gene was assembled in a first round PCR, using 2 mM Tris (pH8.4), 5 mM KCl, 7.5 mM MgCl₂, 1.5 mM dNTP, 2 units of Platinum Tag polymerase (Invitrogen), oligonucleotides V_{H}1, 2, 3, 4, 5 and 6 (1 pmol each) as templates, 5′ GGCAAACCCGGGAGTGGTGA (SEQ ID NO: 32) (50 pmol) as forward primer and 5′GCCACTCGAGCTATTAGTGATGGTGATGGTGGTGAGACGAGACGGTGACCAG (SEQ ID NO: 33) as reverse primer (50 pmol). The PCR was performed for 30 cycles of 94° C. for 30 seconds, 56° C. for 30 seconds and 72° C. for 60 seconds, followed by 72° C. for 10 minutes.

Genetic construction of the variant D2E7 SCA genes encoding the variant SCA proteins of Table 1 was accomplished by site directed mutagenesis. For example, 2-7-SC-5 is a mutant of D2E7 SCA (2-7-SC-1) with an amino acid change from serine to cysteine at the residue 109 in the 218 linker. This gene was created by two rounds of PCR using EN446 DNA as a template and four oligonucleotides as primers.

The primers for construction of clone 2-7-SC-8 were designed as follows:

| | | |
|---|---|---|
| Forward primer 1: | CTCGAATTCACCATGAGATTTCCTTC | (SEQ ID NO: 37) |
| Forward primer 2: | AAGGTGGAAATCAAAGGCTGTACTAGTGGTAGCGGCAAACCC | (SEQ ID NO: 38) |
| Reverse primer 1: | GGGTTTGCCGCTACCACTAGTACAGCCTTTGATTTCCACCTT | (SEQ ID NO: 39) |
| Reverse primer 2: | CGAGAATTCTCATTAATTGCGCAGGTAGCC | (SEQ ID NO: 40) |

Two fragments were amplified separately in the first round of PCR by two primer combinations (forward primer 1 and reverse primer 1, and forward primer 2 and reverse primer 2, 50 pmol each), using 2 mM Tris (pH8.4), 5 mM KCl, 7.5 mM MgCl₂, 1.5 mM dNTP, 2 units of Platinum Tag polymerase (Invitrogen), and 2-7-SC-8 DNA (10 ng) as template. The D2E7 SCA gene variant for 2-7-SC-8 was completed by hybrid extension in the second round of PCR, using forward primer 1 and reverse primer 2 (50 pmol each), 2 mM Tris (pH8.4), 5 mM KCl, 7.5 mM MgCl₂, 1.5 mM dNTP, 2 units of Platinum Tag polymerase (Invitrogen), and the two fragments (10 ng each) from the first round of PCR as templates.

The complete PCR product of the 2-7-SC-8 SCA gene, with cysteine at the residue 109, was cloned into vector pHilD2 and used to transform Pichia GS115 strain, as described below.

The remaining genes of Table 1 were generated by similar site directed mutagenesis steps. For the 2-7-SC-2 gene (SEQ ID NO: 2), the PCR reverse oligonucleotide primer encoded the cysteine codon TGC after the six C-terminal histidine codons. For the 2-7-SC-3 gene (SEQ ID NO: 3), the PCR reverse oligonucleotide primer encoded the cysteine codon TGC after the six C-terminal histidine codons. For the 2-7-SC-4 gene (SEQ ID NO: 4), the PCR reverse oligonucleotide primer encoded the cysteine codon TGC directly after the C-terminal V_{H} serine codon. For the 2-7-SC-6 gene (SEQ ID NO: 6), the central oligonucleotide primer encoded the cysteine codon TGC at position 2 of the 218 linker, and the C-terminal reverse primer encoded the cysteine codon TGC after the six C-terminal histidine codons. For the 2-7-SC-7 gene (SEQ ID NO: 7), the central oligonucleotide primer encoded the cysteine codon TGC at position 2 of the (GGGGS)₃ linker.

For the 2-7-SC-8 gene (SEQ ID NO: 8), the PCR forward oligonucleotide primer encoded the cysteine codon TGC before the N-terminal V_{L} amino acid Asp, and the PCR reverse oligonucleotide primer encoded the cysteine codon TGC after the six C-terminal histidine codons. For the 2-7-SC-9 gene (SEQ ID NO: 9), a 5 codon linker encoding GGGGS replaced the 18 codon 218-linker.

For assembling the complete D2E7SCA gene and expression of D2E7SCA in Pichia, a signal sequence was added at the 5′ end of D2E7SCA gene in a second round of PCR, using 2 mM Tris (pH8.4), 5 mM KCl, 7.5 mM MgCl₂, 1.5 mM dNTP, 2 units of Platinum Tag polymerase (Invitrogen), first round of PCR products of V_{L} and V_{H} of D2E7SCA gene (1 ng each) as templates,

| | |
|---|---|
| 5′CCTCGGAATTCACCATGAGATTTCCTTCAATTTTTACTGCTGTTTTATTCGCAG | (SEQ ID NO: 34) |
| CATCCTCCGCATTAGCTGCTGACATCCAGATGACCCAG (50 pmol) | |
| as forward primer and | |
| 5′CGCGGAATTCTATTAGTGATGGAGATGGAGGAGAGACGAGACGGTGACCAG | (SEQ ID NO: 35) |
| (50 pmol) as reverse primer. | |

The PCR was performed for 30 cycles of 94° C. for 30 seconds, 56° C. for 30 seconds and 72° C. for 60 seconds, followed by 72° C. for 10 minutes.

The gene product of the second round of PCR-assembled D2E7 SCA with 5′ end signal sequence was purified by 1% agarose gel, digested by EcoR1 at 37° C. for 60 minutes and ligated at EcoR1 site of vector pHilD2 (Invitrogen) using T4 DNA ligase at 12° C. for 60 minutes. 100 μl of DH5α competent cells (Invitrogen) were transformed by the ligation reaction product and incubated on ice for 30 minutes and at 42° C. for 45 seconds, then 1 ml of S.O.C media was added and incubated at 37° C. for 50 minutes with shaking at 250 rpm. 0.1 ml of the transformation mixture was spread onto LB Ampicillin (10 mg/L) plates and incubated at 37° C. for 16 hours.

Several pHilD2-D2E7SCA plasmid-transformed DH5α clones on the LB Ampicillin (10 mg/L) plates were grown in 2 ml of LB media at 37° C. for 16 hours. The plasmid mini-preparations of D2E7SCA from each clone were prepared. DNA sequence of pHilD2-D2E7SCA plasmid was confirmed using BigDye terminator cycle DNA sequencing kit (Applied Biosystem) by ABI Prism 310 Genetic Analyzer.

Each of the variant SCA clones listed in Table 1 was generated by the following procedures. For Pichia transformation, pHilD2-D2E7SCA plasmid was digested with Sal1 at 37° C. for 60 minutes and re-suspended in 10 μl distilled water after phenol extraction and ethanol precipitation. Pichia GS115 cells were grown in 50 ml of YPD media at 30° C. for 16 hours with shaking at 250 rpm (OD₆₀₀=1.2), washed in ice-cold distilled water three times and in 1M Sorbitol one time, and finally re-suspended in 0.1 ml of 1M sorbitol.

The prepared Pichia GS115 cells were mixed with Sal1-digested 10 μg of pHilD2-D2E7SCA plasmid in an ice-cold 0.1 cm electorporation cuvette and pulsed under the conditions of 800V, 10 μF and 129Ω by electro cell manipulator (BTX). After pulsing, 1 ml of ice-cold 1 M sorbitol was added into the electroporation cuvette. The whole content was transferred into a 15 ml tube and incubated at 30° C. for 1 hour. 0.2 ml of the transformation mixture was spread onto RDB media plates and incubated at 30° C. for four days.

Several pHilD2-D2E7SCA plasmid-transformed Pichia clones from RDB plates were inoculated into 25 ml of BMGY media in 500 ml flasks and incubated at 30° C. with shaking at 250 rpm for two days. The cells were harvested by centrifugation at 3,000 rpm at room temperature, re-suspended into 5 ml of BMMY of media in 50 ml flasks to induce expression and incubated at 30° C. with shaking for another two days.

1 ml sample from each culture was transferred into micro-centrifuge tubes and centrifuged at 14,000 rpm for 2 minute at room temperature. 40 μl sample from supernatant of each culture was analyzed by Coomassie Blue-stained SDS-PAGE and Western blot.

### Example 2

Recombinant Expression and Purification of SCA Proteins

The SCA proteins described in Example 1 (Clone numbers 2-7-SC-1 through-9). were all produced by expression and secretion from the yeast Pichia pastoris. The secretion signal sequence from the yeast alpha mating factor was inserted directly in front of the mature coding sequence for each of these SCA proteins.

The amino acid sequence of this signal peptide is Met Arg Phe Pro Ser Ile Phe Thr Ala Val Leu Phe Ala Ala Ser Ser Ala Leu Ala \hat{ } Ala (SEQ ID NO: 36) where the \hat{ } indicates the cleavage site. We also included this 20^{th} amino acid of the signal (the alanine after the \hat{ }) in our constructions. Therefore, the amino terminus of each respective mature SCA protein contains this alanine followed by the complete amino acid sequences recorded in FIGS. 1A-1F. N-terminal protein sequence analysis confirmed these correctly processed sequences. These mutant SCA genes were individually ligated at the EcoRI site into the Pichia transfer plasmid pHIL-D2 (Invitrogen Corp.) and transformed into the yeast Pichia pastoris host GS-115. Detailed protocols for these procedures are presented in the Pichia Expression Kit Instruction Manual Cat. No. X1710-01 (1994) from Invitrogen Corporation, incorporated by reference herein. Initial evaluation of expression was done by Coomassie Blue staining of SDS-PAGE gels.

Fermentation of D2E7 SCA Clones

All expression clones of anti-TNFα SCA D2E7, including clones 2-7-SC-2, 2-7-SC-3, 2-7-SC-4, 2-7-SC-5 and 2-7-SC-7,. were generated in Pichia pastoris, a methylotrophic yeast, and proteins were secreted into growth medium. The high density fermentation of D2E7 SCA variants were performed in BMGY medium, the basal medium supplemented with YNB and Biotin (see. Medium Composition) using automatic feed control fermenters (Models: BioFlow 3000 and BioFlow IV; New Brunswick Scientific, Co, Edison, N.J., USA).

A representative profile of fermentation included (a) inoculation of BMGY medium with expression clone, 5% of the fermentation volume, (b) growth phase in BMGY for 16-20 h, (c) growth phase in glycerol (50%) for 4 h, and (d) induction of D2E7 variants with methanol for 45 h. Feeding of each component was optimized with respect to dissolved oxygen level which was set at 30%. The growth temperature was set at 29° C. and pH was maintained at 6.0 using ammonium hydroxide and phosphoric acid during the run. Different parameters monitored over a 68 h fermentation period where OD₆₀₀ values of the growing culture reached from 0.5 to 125 at the end of phase (b), from 125 to 200 at the end of glycerol-feeding phase (phase c) and finally, from 200 to 300 at the end of induction phase (phase d).

On an average, the expression of D2E7 variants in the fermentation supernatants was between 50 and 100 mg/L. 2-7-SC-2, the V_{L}-V_{H} variant with a free cysteine engineered at the carboxyl terminal followed by a histidine tail, was found to be the most robust clone that performed well with excellent reproducibility during fermentation.

Description of Supplies

BMGY (per L)

Yeast Extract:

Peptone:

Glycerol:

Phosphate Buffer:

(1M,pH 6.0)

YNB:

Biotin:

FMT30 (Breox):

Inducer

Methanol

Oxygen

Compressed oxygen

The SCA proteins of Example 1 were purified from Pichia pastoris supernatants to greater than 95% purity by simple two or three column chromatography protocols.

Purification of Proteins from Clones 2-7-SC-2, 2-7-SC-3, 2-7-SC-5, 2-7-SC-7 D2E7 SCAs with Histidine Tag

The D2E7 variants, 2-7-SC-2; SCA2-7-SC-3, SCA2-7-SC-4, SCA2-7-SC-5, and 2-7-SC-7 were purified from fermentation supernatant using a combination of ion-exchange and affinity column chromatography. The supernatant was diafiltered extensively to concentrate the sample while exchanging the spent medium with Buffer C containing Tris, 20 mM at pH 7.4 and 50 mM NaCl. The buffer exchanged sample was then subjected to DEAE column chromatography (Cat #17-0709-01; Amersham BioSciences, Piscataway, N.J.). D2E7 SCAs did not bind to DEAE column under the specified conditions. The flow through from the DEAE column was dialyzed against buffer A containing Tris, 50 mM at pH 8.0 and 0.3 M NaCl and applied to Ni-NTA resin (Qiagen, Inc., Valencia, Calif.) previously equilibrated with the same buffer. Non-specific binding was disrupted using a stringent wash with buffer B containing 10% glycerol in buffer A followed by the removal of glycerol by buffer A. Further low-affinity interactions were washed-off by passing 3 column volumes of 60-100 mM imidazole (100 mM only for EN450). Finally, the bound SCA was eluted from the Ni-NTA column with 3-5 column volume of 250 mM imidazole. For both washing and elution, imidazole was prepared in buffer A.

The peak fractions were pooled and dialyzed extensively against buffer C, the DEAE-buffer. The dialyzed sample was clarified by high speed centrifugation and passed through DEAE column as the final step of purification. Protein concentration of the purified sample was then determined by UV₂₈₀ and by BCA method before storing at −20° C. for future use.

Purification of Proteins 2-7-SC-4 D2E7 SCA without Histidine Tag

Protein L strongly binds to V_{L} domains of many different species. The binding is extremely species specific, and also sub-type specific. The V_{L} domain of 2-7-SC-4 could be recognized by protein L and taking advantage of this specificity of interaction, we purified EN454 in single step, directly from the diafiltered samples. Low pH elution of 2-7-SC-4 from protein L column (Cat # CLBL 201-5, CBD Technologies Ltd, Buffalo, N.Y.) did not affect the structural or functional integrity of the SCA. In brief, fermentation supernatant was diafiltered and exchanged with PBS for loading the sample onto the column. Non-specifically bound proteins were washed away with a large volume of PBS after loading.

The SCA protein was eluted from the column with Glycine buffer (10 mM) at pH 2.0 and collected immediately on 3M Tris to neutralize the solution. Fractions were analyzed on SDS-PAGE, positive fractions were pooled together and dialyzed against PBS. The SCA protein was clarified by high speed centrifugation after dialysis, protein concentration was determined and stored at −20° C. for future use.

Alternate, less preferred methods, like HS (POROS 50 HS, code: 1-3359-07; Applied BioSciences, Foster City, Calif.) or Q-sepharose FF (cat #17-0510-01, Amersham BioSciences, Piscataway, N.J.) were also used effectively in SCA purification.

The following table shows purification of 2-7-SC-2 D2E7 variants by various columns, percentage yields after each step of purification and quality of purification for each variant.

TABLE 3
PURIFICATION
2-7-SC-2 (VL-VH, 218-LINKER, C-CYS, HIS-TAG)

| | Concentration of SCA | Total SCA | |
|---|---|---|---|
| Sample | (mg/ml) | (mg) | Yield (%) |
| Fermentation | 0.07 | 250 | 100 |
| Supernatant | | | |
| Diafiltered | 0.38 | 230 | 92 |
| Sample | | | |
| DEAE-Flow | 0.13 | 210 | 84 |
| Through | | | |
| Ni-Column- | 5.0 | 200 | 80 |
| Purified | | | |
| DEAE-Purified | 2.13 | 190 | 76 |
| Buffer | 2.7 | 186 | 74 |
| Exchanged | | | |

FIGS. 2A and 2B presents representative SDS PAGE analysis of samples from each purification step of 2-7-SC-2 SCA protein. The gel was stained with Coomassie Blue. Purity of the final sample was estimated at 95% from gel densitometry scanning. A small amount of 54 kDa disulfide-linked dimer was visible in the stained gel.

### Example 3

Stability and Reactivity of Maleimide Derivatives of Polyethylene Glycol

Maleimide Reactivity with Amino Acid Residues

Confirmation of the specific reactivity of the maleimide polymers with free cysteine, but not with lysine or histidine, was accomplished by reaction of these compounds with these respective free amino acids. As shown in FIGS. 4A-4C, cysteine, but not histidine or lysine (not shown), is highly reactive with the PEG-maleimides under the standard reaction conditions employed.

Analysis and Stability of Active Maleimide Group

Functional group analysis was conducted in two steps, as follows.

reaction of MAL-PEG with cysteine and

determination of remaining unreacted cysteine after the reaction by titration with 5,5′-dithio-bis(2-nitrobenzoic acid) (“DTNB”).

Determination of active MAL-PEG was conducted at a reaction molar ratio of 1:3 (MAL-PEG:Cysteine) in 50 mM sodium phosphate, pH 6 and 1 mM EDTA.

The reaction was conducted as follows.

A 1/40 volume of cysteine in H₂O was added to a 1 mM PEG solution to a final concentration of 3 mM. The mixture was incubated at 25° C. in the dark for 10 min, followed by DTNB titration. In DTNB titration, a 1/50 volume of the reaction mixture containing the cysteine and Cys-MAL-PEG mixture was added to 0.2-0.3 mM DTNB in 100 mM sodium phosphate, pH 7.3 and 1 mM EDTA,.

The final concentration of remaining cysteine was between 0.04-0.06 mM. Absorbance at 412 nm was recorded after 5 min of equilibrium at 25° C. using 13,300 M⁻¹ .cm⁻¹ as an extinction coefficient of DTNB. The reaction of MAL-PEG with Beta-mercaptoethylamine was also investigated. This reaction is not quantitative because beta-mercaptoethylamine is air-sensitive and hygroscopic. The stability of MAL-PEG was monitored by a UV scan between 240 and 400 nm. MAL-PEG had a maximum absorbance at 300 nm. The peak disappeared after reacting with cysteine or hydrolyzing in the presence of 0.1N NaOH at 37° C. for 2 hrs.

Stability of MAL-PEG

The stability of MAL-PEG is dependent on pH, temperature, and incubation time. It was considered stable if there was less than 10% decrease in absorbance at 300 nm. 1 mM MAL-PEG is stable at 4° C. for at least 24 hrs in all buffers tested (pH 5, pH 6, and pH 7 phosphate buffers). At 25° C. and pH 5.0, MAL-PEG is stable for 33 hours.

Specificity of Mal-PEG Reaction with Cysteine

The reaction with Cysteine was completed in less than 2 min regardless of reaction pH (5, 6, or 7) and temperature (4° C. or 25° C. ).

The reaction with lysine (Lys:MAL-PEG=15:1) was not observed in pH 5 and pH 6 buffers during 24 hrs incubation at 4° C. or 25° C. However, at pH 7, 25° C., greater than 10% MAL-PEG reacted with lysine during 24 hours of incubation.

There was no reaction with histidine at a molar ratio of 15:1 (Histidine:MAL-PEG) pH 5, 6, and 7 during 24 hours incubation at 4° C. or 25° C.

### Example 4

PEGylation of SCA Proteins

4A. Materials and Methods

HiPrep® 26/10 and G-25 PD-10 desalting columns (Pharmacia Biotech, 17-1408-01, New Jersey) and Poros 50 Micron HS media (Applied Biosystems) were used. mPEG-maleimide compounds were purchased from Nektar Therapeutics (San Carlos, Calif.; formerly Shearwater Corp.) or synthesized at Enzon Pharmaceuticals, Inc.

PEG-MAL polymers employed in this study included the 40 kDa branched PEG2, 20 kDa linear PEG, 5 kDa linear PEG, 20 kDa bis-MAL bifunctional PEG, and 40 kDa branched U-PEG. N-ethylmaleimide and 6-(Biotinamidocaproylamido) caproic acid N-Hydroxysuccinimide ester were purchased from Sigma. rProtein A Sepharose Fast Flow was obtained from Amersham Biosciences Corp. (Piscataway, N.J.). Ultralink Iodoacetyl® was obtained from Pierce Biotechnology, Inc (Rockford, Ill.). DMSO, (Minneapolis, Minn.). Streptavidin-Phycoerythrin was obtained from BD Sciences (San Jose, Calif.). The 96 well microtiter plates were purchased from Midwest Scientific (St. Louis, Mo.).

Streptavidin-peroxidase was from Sigma and TMB peroxidase substrate was from Moss, Inc. (Pasadena, Md.). TNFα was purchased from Chemicon (Temecula, Calif.). Titrisol® iodine solution was obtained from EM Science (Gibbstown, N.J.)

4B. Reduction of D2E7 SCAs

The free cysteine residue at the C-terminus or linker of the SCAs isolated by Example 3 was reduced before reaction with MAL-PEG. The reduction solution contained 3 mg/ml D2E7 SCA, 2 mM dithiothreitol (DTT), 2 mM EDTA, and 100 mM sodium phosphate, pH 7.8. The reduction was conducted at 37° C. for 2 hrs. Free DTT was removed on HiPrep® desalting column for 15-ml samples, or PD-10 for a 4-ml sample. The column was equilibrated with 100 mM sodium phosphate pH 6.0, 2 mM EDTA. The recovery of D2E7 SCA after reduction and desalting was 85%. Other reductants, including beta-mercaptoethylamine and beta-mercaptoethanol, were also successfully used in modified procedures. Sulfhydryl group quantitation was performed as described by Grassetti D R et al, 1967, Archives Biochem Biophys 119: 41-49, and Riddles P W et al, 1979, Anal Biochem 94: 75-81, incorporated by reference herein. Near quantitative reduction of one thiol per SCA was achieved.

4C. PEGylation and Purification of SCA Methods

The SCA proteins isolated by Example 3 were PEGylated through cysteine-specific reactions with PEG maleimide compounds. 2-7-SC-2 and 2-7-SC-5 were chosen for extensive studies of PEG-SCA characterization. For these SCA proteins, maleimide-PEG conjugates with 5 kDa, 20 kDa, 40 kDa (branched) and bis-maleimide compounds were examined. Reaction of the SCA proteins with N-ethylmalemide provided a control conjugation reaction which blocks the free thiol but adds minimal molecular mass. Other D2E7 SCA proteins were modified with selected PEG-maleimide polymers as listed in the section on BIAcore analysis.

The reaction buffer contained 1 mg/ml reduced SCA, 100 mM sodium phosphate pH 6.0, 2 mM EDTA, and PEG maleimide compound at a reaction molar ratio of 10:1 (PEG:D2E7). The reaction was conducted at 25° C. under Nitrogen for 2 hrs.

The typical yield of the conjugation analyzed on SDS-PAGE was 80%. An HS column was used for purification of PEG-SCA from native SCA, high molecular weight impurities, free PEG, side reaction products, and endotoxin. In less preferred methods, S and SP columns were also successfully utilized. The column equilibration buffer contained 10 mM sodium phosphate, pH 5.0, and elution buffer was made of 1 M NaCl in 10 mM sodium phosphate, pH 5.0. Free PEG was in the flow through. PEG-D2E7 SCA conjugates were eluted sequentially with conjugates with higher numbers of attached PEG eluting first, followed by conjugates with a single polymer attached, and finally, native D2E7 SCA. PEG-D2E7 conjugates of different sizes were therefore eluted at different concentrations of NaCl.

4D. Reduction of D2E7 SCA—Results Summary

D2E7 SCA produced in Pichia pastoris and purified as described has to be reduced prior to a reaction with MAL-PEG.

DTT at the concentrations from 0.5 mM to 50 mM was tested. It was shown that 0.5 mM was sufficient to reduce monomer to dimer. DTT at a concentration higher than 10 mM generated some precipitate. The higher the concentration of DTT used, the higher amount of D2E7 SCA precipitated. The precipitate might be denatured D2E7 SCA. 2-mM DTT was chosen for standard PEGylation protocols.

Beta-mercaptoethylamine, glutathione, and cysteine from 2 mM to 10 mM were also investigated. It was shown that a concentration higher than 10 mM was required to reduce the dimer to monomer.

DTT at 0.5 mM was as efficient, as the other reducing reagents at 10 mM, in reduction of the 2-7-SC-2 dimer to monomer.

The yields of conjugates after reduction were about 80% of the starting reduced SCA protein.

4E. PEGylation—Results Summary

The reaction ratios ranging from 1:1 to 10:1 (MAL-PEG:D2E7 SCA) were investigated for PEGylation yield at pH 6.0. A ratio of 1:4 was the minimum required to give a high yield of the conjugate.

Reaction times from 10 min to 24 hrs at 25° C., and 18 hrs at 4 ° C. were studied. It was shown that the reaction was completed in 10 min at 25° C. (the shortest time tested).

A high concentration of protein (e.g., >1 mg/ml) is not desirable for a reaction of the free cysteine residue of D2E7 SCA with MAL-PEG since yields are reduced. This contrasts with the optimal approach for a non-specific multi-PEGylation. Protein concentrations of 0.5, 1.5, 2.0, 2.5, and 3 mg/ml were tested. D2E7 SCA protein of 0.2-1 mg/ml was used as the preferred concentration for constructing the conjugates.

The reaction pH from 5-8 was investigated. For PEGylation, pH 6.0 was used. Unconjugated D2E7 SCA could be recycled for re-conjugation. The yield from the second conjugation reaction was similar to that obtained for the initial D2E7 SCA PEGylation. The best conjugation yield was 85%. Overall, the results demonstrate that monoPEGylated SCA proteins may be generated in good yield via robust conjugation methods using the designed single free-thiol variant SCA proteins.

4F. Purification Results Summary

Ultrafiltration with polyethersulfone membranes cannot be used for concentration and changing buffers of the 2-7-SC-2 SCA protein and its conjugates, since most of the protein was lost to the membrane.

There was 100% recovery of the protein on Millipore regenerated cellulose membranes such as Centriplus, Centricon, and Amicon

There was 10% loss of the protein on a 0.2-μm low protein binding sterile filter.

The total yield after two steps of purification, two steps of concentration, and one step of filtration was 30-40%.

The purified PEG-SCA proteins were subjected to SDS PAGE analysis and visualized with Coomassie Blue stain (data not shown). The analysis indicated that trace amounts (˜1%) of unreacted SCA protein remained in the purified 40 kDa MAL-PEG and kDa MAL-PEG reactions. Iodine stain of SDS PAGE gel, which highlights the polyethylene glycol containing compounds, also revealed trace amounts (<1%) of free PEG that were detectable in the purified 40 kDa MAL-PEG and 20 kDa MAL-PEG reactions.

The 40 kDa MAL-PEG reactions sometimes also displayed a trace (˜1%) of very high molecular weight PEG impurities. Polymers in the very high mass range were also detectable in the starting unreacted 40 kDa MAL-PEG polymers. N-ethylmaleimide reduction totally blocks the formation of dimers in the SCA proteins having a single free cysteine.

4G. Removal of Endotoxin by Ion Exchange Chromatography

Endotoxin present in protein samples was removed by DEAE or HS columns. At pH 7-8, endotoxin was bound to a DEAE column while the D2E7 SCA was present in the flow through fraction, whereas at pH 5.0, endotoxin was present in the flow through fraction and D2E7 SCA was bound to HS column. An HS column was used to remove endotoxin from D2E7 SCA protein. The column equilibration buffer contained 10 mM sodium phosphate, pH 5.0 and elution buffer contained 1 M NaCl and 10 mM sodium phosphate, pH 5.0. Typical endotoxin values in the purified samples were below 1 EU/ml.

### Example 5

Analytical Characterization of SCA and PEG-SCA.

5A. Determination of Protein Concentration

Protein concentrations were determined by UV at 280 nm. The extinction coefficient of the SCAs obtained in Example 3 were 1.24 ml/mg.cm. The concentration was also confirmed by the bicinchoninic acid assay (“BCA”), obtained as a Micro BCA Protein Assay Reagent kit from Pierce Biotechnology, Inc (Rockford, Ill.) using lysozyme or a Fab as standards. The BCA assay was conducted as recommended by the manufacturer and essentially according to the method of Smith, P. K., et al. 1985, Anal. Biochem. 150, 76-85, incorporated by reference herein.

Protein Concentration Determination Results

UV at 280 nm (data not shown) and BCA, as discussed supra, using lysozyme and a human Fab as standards gave similar results in protein concentration determinations. For the BCA analysis, EDTA should be removed along with DTT after reduction, since these reagents interfere with the assay.

All samples for animal studies were analyzed for protein content by UV and confirmed by BCA using lysozyme as a standard.

5B. Anti-D2E7 SCA Polyclonal Antibody and Biotinylated Anti-D2E7 SCA Antibody

Purification of Anti D2E7 SCA Antibody.

Anti-2-7-SC-1 SCA antibodies were raised in rabbits and purified by Protein A chromatography and D2E7 SCA-conjugated affinity column chromatography. For a Protein A column purification, the antibody was diluted with two volumes of Tris buffer to make a final concentration of 0.1 M Tris-HCl, pH 8.0, 0.02% NaN₃. The diluted samples were loaded on a 2-ml Protein A Sepharose column which was equilibrated with 0.1 M Tris-HCl, pH 8.0, 0.02% NaN₃ at an equal volume of antiserum to protein A resin. Anti-2-7-SC-2 SCA was eluted out with 50 mM glycine, pH 3.0 to a 1/10 volume of 1 M Tris-HCl, pH 8.0. The antibody concentration was determined at 280 nm using an extinction coefficient of 0.8 ml/mg.cm. A D2E7 SCA-conjugated affinity column was prepared by a coupling reaction of Ultralink Iodoacetyl with the free cysteine residue of 2-7-SC-2 SCA protein. Specifically, ˜7 ml of Ultralink Iodoacetyl resin rinsed with 2 volumes of 50 mM phosphate, 5 mM EDTA, pH 7.8, were mixed with 6.5 ml of 1.45 mg/ml 2-7-SC-2 SCA at 25° C. for 15 min. The coupling reaction was monitored by measuring absorbance of the supernatant at 280 nm. The protein concentration in the supernatant was decreased by 80%. The resin was washed with 3 volumes of 50 mM phosphate, pH 7.8, 5 mM EDTA and then treated with 50 mM cysteine for 40 min. The sample was then transferred to the column and washed with 1 M NaCl , 50 mM glycine, pH 3.0, and then with PBS. The anti-2-7-SC-2 SCA antibody purified from the Protein A column was passed through the D2E7 SCA-conjugated column which was equilibrated with standard PBS. The column was chased to baseline with PBS and the antibody was eluted out with 50 mM glycine, pH 3.0 to a 1/10 volume of 1 M Tris-HCl, pH 8.0.

Biotinylated Anti-D2E7 SCA Antibody.

Glycine and Tris components in the samples were removed by a PD-10 desalting column, which was equilibrated with 50 mM phosphate, pH 7.6, 100 mM NaCl. To the antibody solution was added 1/10 volume of activated Biotin in DMSO at a reaction molar ratio of 40:1 (biotin:antibody). After 1 hr at 25° C. , the biotinylated antibody was purified on a PD-10 desalting column which was equilibrated with PBS (10 mM sodium phosphate, 138 mM NaCl, 2.7 mM KCl, pH 7.4).

5C. Western Blot

Anti D2E7 SCA rabbit antiserum was used as a primary antibody and goat anti rabbit HRP was used as a secondary antibody. Binding was measured with a TMBM peroxidase substrate. Rabbit antiserum was also previously prepared from against the synthetic 18 residue 218-linker peptide. Reactivity with SCA proteins containing this linker was also established.

Western Blot Results

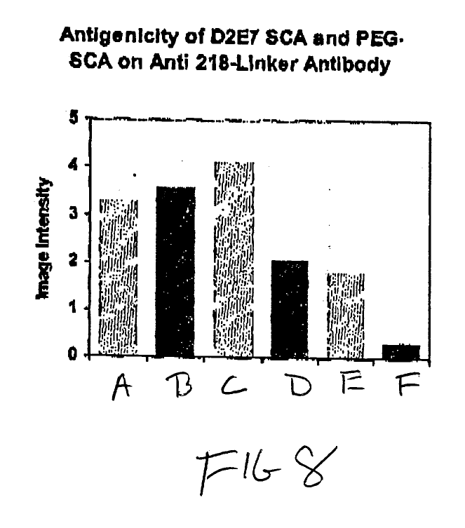

All bands shown on the gels from purified preparations were Western Blot positive. FIG. 7 shows an example of a Western analysis of D2E7 SCA and PEG-SCA compounds detected with anti-2-7-SC-1 antiserum. The primary detection antibody was anti-2-7-SC-1 SCA rabbit antiserum prepare from rabbits immunized with the purified recombinant SCA protein. Lane 1 and 7, molecular weight markers (250, 148, 98, 64, 50, 36, 22, 16, 6 and 4 kDa); lane 2, 2-7-SC-2 SCA protein, lane 3, ethyl-2-7-SC-2 ; lane 4, PEG (5 kDa)-2-7-SC-2 ; lane 5, PEG (20 kDa)-2-7-SC-2 ; lane 6, PEG (40 kDa)-2-7-SC-2. It was not established what proportion of the 2-7-SC-2 SCA protein existed as monomer and dimer in the animal studies because of the low concentration in plasma. However, the slightly more rapid clearance of the 2-7-SC-2 protein modified with N-ethylmaleimide could suggest that some of the starting SCA protein was dimer and exhibited slower clearance due to a larger mass or avidity.

5D. Purity Analysis

The dimer form is generated by cross linking of the free cysteine residues since the 2-7-SC-2 SCA modified with N-ethyl-maleimide did not show any dimer on a non-reducing SDS-PAGE.

The purified PEG-SCA conjugates typically contained essentially no free PEG as detected by Iodine stain, less than 1% of unmodified SCA, and less than 1% high molecular weight molecules as detected on SDS-PAGE.

5E. Iodine stain

SDS PAGE gels were rinsed with dH₂O and placed in 5% barium chloride solution. After 10 min of gentle mixing, the gel was rinsed with H₂O and then placed in 0.1 M Titrisol® iodine solution for color development.

5F. Mass Determination

The exact mass values of SCA and PEG-SCA conjugates were determined by matrix assisted laser desorption ionization mass spectrometry (“MALDI-TOF”; Bruker Daltronics OmniFlex NT) using an internal standard with similar molecular weight on the α-cyano-4-hydroxy cinamic acid (CHCA) matrix. Apparent molecular weights (Stoke radius) of the SCA proteins were estimated using Superdex 200 HR 10/30 Gel Filtration column chromatography [Amersham Biosciences, by the method of the manufacturer] which was equilibrated in 50 mM sodium phosphate, pH 6.5 and 150 mM NaCl,.

Additionally, analysis of molecular masses on 4-20% SDS-PAGE gels was performed using appropriate protein and PEG-protein standards. The apparent molecular weight of the PEG-40k-SCA, as determined by size exclusion chromatography, was 670 kDa, or about 10-fold more than its molecular mass.

Molecular Weight Determination Results for 2-7-SC-2:

The molecular weight determination results are shown in Table 4.

TABLE 4

| Compound | MALDI-TOF | SEC × 10³ | SDS-PAGE × 10³ |
|---|---|---|---|
| D2E7 SCA | 27,394 ± 295 | 20 | 30 |
| Ethyl-D2E7 SCA | 27,858 | 20 | 30 |
| PEG-5k-D2E7 SCA | 33,715 | | 36 |
| PEG-20k-D2E7 SCA | 49,874 | 340 | 56 |
| PEG-40k-D2E7 SCA | 73,123 | 670 | 170 |

The correlation of PEG size over protein size on 4-20% SDS gel was Y=0.00156X Other D2E7 SCA variants and PEG-SCA compounds displayed comparable mass values that conformed to their molecular weight and polymer size.

5G. N-terminal Sequencing and Peptide Mapping:

N-terminal sequencing of 2-7-SC-2 and 2-7-SC-5 confirmed the expected processing of signal sequence, such that the N-terminal amino acid of the secreted SCA proteins is alanine followed by the first residue of V_{L}.

Peptide Mapping on 2-7-SC-5-40 kDa PEG (EN456-40K)

The protein (0.2 mg total) was denatured and reduced in 6 M Guanidine HCl containing 1 mM EDTA and 5 mM DTT. The solution was allowed to incubate at 37° C. for 1 h. Alkylating agent, iodoacetamide, was added to the final concentration of 15 mM and the reaction was performed at room temperature for 1 h. After alkylation, the excessive iodoacetamide was inactivated by adding β-mercaptoethanol to 45 mM (final concentration) and the solution was subjected to PD10 desalting column. The alkylated PEG-2-7-SC-5 (EN456-40K) was concentrated with Centricom 10 and then hydrolyzed by TPCK-treated trypsin with enzyme to protein ratio of 1:20 (w/w). The hydrolysis was allowed for 6-8 h at 37° C. and then added same amount of fresh trypsin for overnight reaction. The hydrolyzed protein solution was brought to dryness by Speedvac and reconstituted in HPLC-grade water.

The resultant peptide mixture was fractionated by HPLC size exclusion chromatography (Superdex 75) with HPLC-grade water. The factions were manually collected and analyzed by Tricine SDS-PAGE stained with iodine solution (20 mM in 5% BaCl). The positively stained fractions were further resolved by reversed-phase HPLC (Jupiter C 18, 2×250 mm) with the gradient of acetonitrile (containing 0.05% TFA) from 5-70% in 60 min. The peaks were collected manually and dried in Speedvac. The peaks were reconstituted with 10 μl water and 5 μl was taken for Tricine SDS-PAGE analysis. The positively iodine-stained fraction (only one, ˜40 min of retention time) was subjected to amino acid sequencer analysis (Applied Biosystems). The sequence obtained from the analysis was G [symbol] TSGSGKPG, where the blank square represents the modified amino acid, indicating that maleimide—PEG 40K is accurately attached to cysteine (position 110 from N-terminal Ala 1).

5H. Stability of D2E7 and PEG-D2E7—Results

D2E7 SCA and PEG-D2E7 SCA conjugates were shown to be stable after 10 cycles of freeze-thaw. Aliquots of native 2-7-SC-2 SCA (concentration 1.1 mg/ml in 50 mM Tris/Glycine buffer at pH 7.0) were subjected to freezing at −80° C. for 15 minutes followed by thawing at 37° C. for 10 minutes. The freeze-thaw maneuver was performed for 3, 5, and 10 cycles and the integrity of the native SCA was analyzed by SDS-PAGE and by established TNF-sensitive cell rescue assay. The SCA was found be to very stable and could physically withstand at least 10 freeze-thaw cycles without showing any degradation as determined by Coomassie blue staining of polyacrylamide gel (data not shown). There was no change in biological activity of 2-7-SC-2 (IC₅₀: 224.6 nM) upon 5 cycles of freeze-thaw.

However, a reduction in TNF-neutralizing efficiency of EN450 was observed after 10 cycles of freeze-thaw (IC₅₀: 302.5 ng/ml). This study evidently limits the freeze-thaw of EN450 for up to 5 cycles while retaining its full biological activity.

All D2E7 PEG-SCA proteins in this study were found to be stable for 30 days at 4° C. in 20 mM sodium phosphate, pH 6.5, 150 mM NaCl. 2-7-SC-2 SCA protein was stable at a pH 3-10, 25 ° C., 18 hrs incubation. NaCl at a concentration up to 1.2 M had no effect on activity or solubility of 2-7-SC-2, in 20 mM sodium phosphate, pH 7.4, at 25° C.

5I. Antigenicity

PEGylated D2E7 SCA proteins display a marked decrease in binding efficiency to anti-D2E7 SCA polyclonal antibodies. PEG-SCA 2-7-SC-5 is marginally reactive with anti-218 peptide rabbit serum as analyzed by Western blots using anti-218 antiserum. FIG. 10 shows the scanned image intensity of the bands from the Western blot on which equal amounts of protein were loaded in each lane.

### Example 6

Flow cytometry Analysis of SCA and PEG-SCA Proteins

Cell Surface Receptor Binding Assay for 2-7-SC-2, 2-7-SC-3, 2-7-SC-4, 2-7-SC-5

The WEHI-13VAR cell line was used to analyze TNF-alpha binding to the cell receptor in the presence of D2E7 SCA or PEG-D2E7 SCA. The Biotin TNF-alpha (0.04 μg) was preincubated with D2E7 SCA or PEG-D2E7 SCA (1-4 μg) in 50 ul of FACS buffer (1% FBS and 0.05% NaN₃ in PBS) at 25° C. for 30 min and then at 4° C. for 15 min with shaking.

At the same time, the controls, such as biotinylated soybean trypsin inhibitor (0.05 μg), polyclonal goat IgG anti-human TNF-alpha antibody (20 ug), and CC49 SCA (4 μg), were also pre-incubated with biotin-TNF-alpha (0.04 μg) in 50 ul FACS buffer. WEHI-13VAR cells were detached from flask using ice cold 20 mM EDTA in PBS, 37° C. for 2-3 min. The cells were resuspended in RPMI 1640 Medium and span down at 2000 rpm for 5 min. The cells were then washed once with medium and twice with FACS wash buffer and counted using a hemacytometer.

The cells were suspended in FACS buffer to a final concentration of 2×10⁶ cells/ml. All amber eppendorf tubes used for cells were blocked with FACS buffer for at least 1 hr at 4° C. For the effect of Actinomycin D and Fe block reagent (BD Biosciences)on TNF-alpha binding to cell receptor, the cells (10⁵) were pre-incubated with 0.05 ug Actinomycin D/1×10⁶ cells or 1 μg Fe blocking/×10⁶ cells in 50 ul FACS buffer for 15-30min at 4° C. To 50-μl. mixture of TNF-alpha and PEG-D2E7 SCA was added 50 ul of 1×10⁵ WEHI-13VAR cells. After 60 min incubation at 4° C. in the dark, the cells were span down and resuspended in 80 μl cold FACS buffer. 10-μl of Streptavidin-Phycoerythrin was added. The mixture was incubated in the dark at 4° C. for 30 min. The cells were then washed twice with cold 1 ml of FACS buffer and resuspended in 0.3 ml FACS wash buffer for analysis.

Flow Cytometry Analysis—Results

Biotinylated soybean trypsin inhibitor, polyclonal goat anti-human interferon-alpha IgG, and CC49 SCA (Enzon) exhibited no binding and served as negative controls. Preincubation with Fc block reagents (BD Biosciences) and Actinomycin D to the cells had no effect on TNF-alpha Binding.

PEG-D2E7 2-7-SC-2 SCA conjugates (ethyl-, 5 k, 20 k or 40 k PEG) completely eliminated TNF-alpha binding to the cells at a molar ratio higher than 16:1 (D2E7 SCA:TNF-alpha).

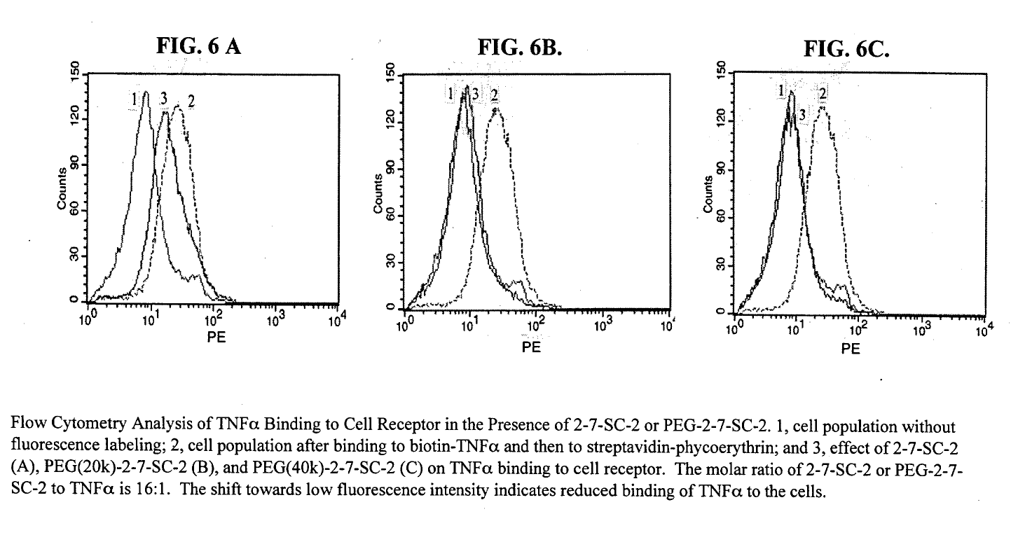

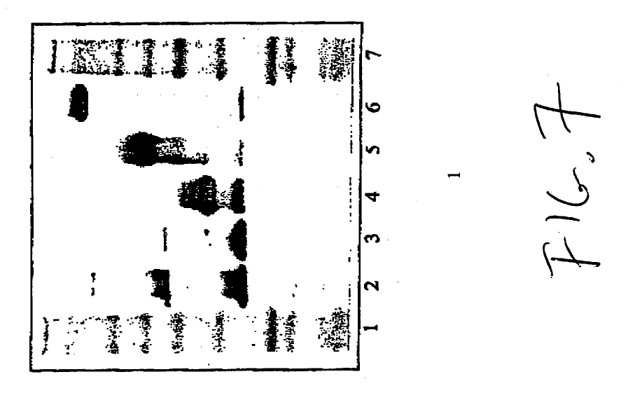

At the same molar ratios of D2E7 SCA to TNF-alpha, native D2E7 SCA also reduced TNF-alpha binding to the cells, but not fully. Therefore, in this analysis, the PEG-SCA versions of the D2E7 were more potent than the native SCA proteins. FIGS. 6A, 6B and 6C shows representative data of 2-7-SC-2 SCA and PEG-SCA compounds in flow cytometry analysis of the capacity of these compounds to prevent biotin labeled TNF-α from binding to its receptor on WEHI-13VAR cells. These data show that the anti-TNF-α PEG-SCA compounds are highly active in blocking the binding of this cytokine to its receptor in a cell based system.

Flow Cytometry Analysis of TNFα Binding to Cell Receptor in the Presence of 2-7-SC-2 or PEG-2-7-SC-2. 1, cell population without fluorescence labeling; 2, cell population after binding to biotin-TNFα and then to streptavidin-phycoerythrin; and 3, effect of 2-7-SC-2 (FIG. 6A), PEG(20 k)-2-7-SC-2 (FIG. 6B), and PEG(40k)-2-7-SC-2 (FIG. 6C) on TNFα binding to cell receptor. The molar ratio of 2-7-SC-2 or PEG-2-7-SC-2 to TNFα is 16:1. The shift towards low fluorescence intensity indicates reduced binding of TNFα to the cells.

### Example 7

BiaCore Analysis

Kinetic Analysis of the interaction of Recombinant hTNF-alpha with D2E7 SCA and PEG-SCA

The interaction between TNF-α and D2E7 SCA variants and their PEGylated forms was analyzed by surface plasmon resonance (SPR) techniques using a BiaCore X instrument (BiaCore, Inc.; Piscataway, N.J.). Recombinant human TNF-α of >97% purity (Pierce; Rockford, Ill.) was immobilized on a CM5 chip (BiaCore, Cat # BR-1000-14) as a 10 μg/ml solution at pH 5.0 (acetate buffer, BiaCore, Cat t # BR-1003-51). The immobilized surface was washed three times with acetate buffer, pH 4.5 (BiaCore, Cat # BR-1003-50) and subjected to ligand stability analysis for 6 cycles with 500 nM native SCA.

D2E7 SCA served as analyte with acetate at pH 4.5 as the regeneration buffer. Over the stable TNF-α-bound surface, different concentrations of SCAs or PEG-SCAs were examined for association (3 minutes) and dissociation (2 minutes or 5 minutes) and the data were analyzed for kinetic parameters (e.g., kₒₙ, k_{off}, K_{A}, and K_{D} values) using BiaEvaluation software (version 3.0). HBS-N (BiaCore, Cat t # BR-1003-69) was used as the running buffer in this protocol.

Kinetic Analysis of Interaction Between Immobilized rhTNF-α with D2E7 SCA or PEG-SCA

Methods

TNF-alpha

Source: Pierce, recombinant form, cat # RTNFA50

MW: 17.4 k D, 157 aa

Purity: >97%

Reconstitution: In distilled water “DW” to a concentration of 100 μg/ml. No additives are present in the preparation

Storage: Stored at −70 degrees C.

D2E7 SCA: Clone 2-7-SC-2

Source: Enzon Pharmaceuticals, Inc., recombinant form, expressed in Pichia

MW: 27 kD, with 218 linker

Purity: >90%

Reconstitution: In 10 mM phosphate buffer, pH 7.0, with 150 mM NaCl

Storage: Stored at 4.0 degrees C.

Immobilization: Using New CM5 Chip

TNF conc.: 10 μg/ml, diluted directly from the stock in acetate buffer at pH 5.0

Flow rate: 5.0 μl/min

Channels: FC1-2, 3.0 min activation with 1:1 NHS/EDC mixture

Channel: FC2, manual injection of 15 μl of TNF, 10 μg/ml (Less Volume to be Injected to Achieve Lower RUs)

Channels: FC1-2, 3 minutes inactivation with 1 M Ethanolamine, pH, 8.5

Channel: FC1-2, manual injection of 25 μl of BSA, 1 μg/ml in HBS-N buffer

Channels: FC1-2, 1 min injection of 10 mM acetate, pH 4.5, 100 μl/min, to clean the injection port

Final RU (response unit) was 199.

The CM5 Chip was washed with HBSN buffer and tested for at least 6 cycles of stability with 500 nM 2-7-SC-2. The CM5 Chip was then used for kinetic analysis.

Kinetic Analysis of D2E7 2-7-SC-2

Concentration of 2-7-SC-2: diluted in HBS-N immediately before injection

2.98 μg/ml (1080 nM)

1.49 μg/ml (540 nM)

0.745 μg/ml (270 nM)

0.3725 μg/ml (135 nM)

0.186 μg/ml (67.5 nM)

93 ng/ml (33.75 nM)

46.5 ng/ml (16.875 nM)

23.28 ng/ml (8.4375 nM)

0 μg/ml (0 nM)

Flow Rate: 25 μl/min (Note: 30 μl/min resulted in similar binding as that of 20 and 25 μl/min)

Duration of association: 3 minutes

Duration of dissociation: 2 minutes and 5 minutes

Regeneration Buffer: 10 mM Acetate, pH 4.5

Regeneration was performed in two steps, 100 μl wash at 100 μl/min as the 1^{st} wash and 40 80 ul at 100 μl/min as 2^{nd} wash, depending on the RU left on the chip after the 1^{st} wash

Data Analysis

The association and dissociation kinetic curves for the bimolecular binding reaction were analyzed using 1:1 binding fit with and without mass transfer limitations. No significant improvement in kinetics was achieved by including mass transfer parameters, showing that mass transfer phenomenon was not prevalent in the experiments. 2-7-SC-4 and PEG-40k-2-7-SC-4 were prepared for this purpose FACS results independently showed that there was no difference between D2E7/PEG-D2E7 SCA binding to TNF-alpha, with and without the SCA his-tag segment.

The results from Biacore and cell rescue studies have also confirmed that the his-tag segment is not responsible for binding events of the antigen and SCA.

Direct binding kinetics were determined by immobilizing TNF-alpha on CM5 Chip and allowing different concentrations of native and PEG-versions of 2-7-SC-2 to flow over the bound ligand. Table 5, below, provides the ka (kₒₙ), kd (k_{off}), K_{A}, and K_{D} values of different forms of 2-7-SC-2 SCAs.

Kinetic Parameters of 2-7-SC-2 SCA Compounds

TABLE 5

| 2-7-SC-2 | | | | |
|---|---|---|---|---|
| Versions | ka (M⁻¹s⁻¹) | kd (s⁻¹) | KA (M⁻¹) | KD (M) |
| 2-7-SC-2-native | 3.28e5 | 3.92e−4 | 8.36e8 | 1.2e−9 |
| 2-7-SC-2-NE- | 1.47e5 | 7.87e−5 | 1.86e9 | 5.37e−10 |
| mal | | | | |
| 2-7-SC-2-5K- | 4.96e4 | 3.01e−4 | 1.65e8 | 6.06e−9 |
| PEG | | | | |
| 2-7-SC-2-20K- | 1.6e3 | 4.18e−4 | 3.83e6 | 2.61e−7 |
| PEG | | | | |
| 2-7-SC-2-40K- | 4.47e3 | 6.78e−4 | 6.59e6 | 1.52e−7 |
| PEG | | | | |

Table 5, above, confirms that the PEGylated SCA proteins maintain high affinity for their ligand. However, different PEG-SCA designed molecules show differences in on-rates and off-rates. In particular, the 40 kDa PEG version of 2-7-SC-2 has significantly diminished on-rates, but retained off-rates, when compared to the parent SCA. This could reflect a steric hindrance effect in this artificial binding environment on the BIACore chip by the large and flexible PEG polymers. In contrast, the cell based assays described elsewhere in this study show a similar binding potency for the native and 40 kDa PEGylated SCA proteins.

The specific trends in on-rate and off-rate perturbations by PEGylation could reveal a compound-specific conformation or arrangement of the polymer with respect to the conjugated protein. The further studies on additional PEG-SCA compounds described below highlight this possibility. 2-7-SC-4-40 kDa-PEG data indicate that placement of the PEG polymer directly at C-terminus substantially improved off-rates. The strategy of using the defined parameters of SCA cysteine placement and PEG polymer mass as disclosed in this study may allow the optimization of binding and activity properties for any individual PEG-SCA protein conjugate.

Binding Kinetics of 2-7-SC-5 and 2-7-SC-7 to rhTNF

Direct binding kinetics were determined by immobilizing TNF-alpha on CM5 Chip and allowing different concentrations of native and PEG-versions of 2-7-SC-5 and 2-7-SC-7/to flow over the bound ligand. Tables below provide the ka, kd, K_{A}, and K_{D} values of different forms of 2-7-SC-5 and 2-7-SC-7 .

TABLE 6
KINETIC PARAMETERS OF 2-7-SC-5 SCA COMPOUNDS

| 2-7-SC-5 Versions | Ka (M⁻¹s⁻¹) | kd (s⁻¹) | KA (M⁻¹) | KD (M) |
|---|---|---|---|---|
| 2-7-SC-5-native | 1.73e5 | 1.12e−5 | 1.55e10 | 6.44e−11 |
| 2-7-SC-5-40K-PEG | 2.04e3 | 2.23e−6 | 9.18e8 | 1.09e−9 |

TABLE 7A
KINETIC PARAMETERS OF 2-7-SC-7

| Version | Ka (M⁻¹s⁻¹) | kd (s⁻¹) | KA (M⁻¹) | KD (M) |
|---|---|---|---|---|
| 2-7-SC-7-native | 3.51e4 | 2.96e−6 | 1.19e10 | 8.43e−11 |

TABLE 7B
KINETIC PARAMETERS OF 2-7-SC-7

| 2-7-SC-7 Version | ka (M⁻¹s⁻¹) | kd (s⁻¹) | KA (M⁻¹) | KD (M) |
|---|---|---|---|---|
| 2-7-SC-7-20K-PEG | 4.37e4 | 2.89e−4 | 1.51e8 | 6.61e−9 |

Direct binding kinetics were determined by immobilizing TNFα on CM5 Chip and allowing different concentrations of native and PEG-versions of EN452/460 to flow over the bound ligand. Tables below provide the ka, kd, K_{A}, and K_{D} values of different forms of 2-7-SC-3 and 2-7-SC-7.

TABLE 8A
Kinetic Parameters of 2-7-SC-3 SCAs and Conjugates

| 2-7-SC-3 Versions | ka (M⁻¹s⁻¹) | kd (s⁻¹) | KA (M⁻¹) | KD (M) |
|---|---|---|---|---|
| 2-7-SC-3-native | 4.64e4 | 4.16e−4 | 1.1e8 | 9.05e−9 |
| 2-7-SC-3-20K-PEG | 1.02e4 | 2.5e−4 | 4.07e7 | 2.45e−8 |
| 2-7-SC-3 40K-PEG | 4.14e3 | 4.04e−4 | 1.03e7 | 9.74e−8 |

TABLE 8B
Kinetic Parameters of 2-7-SC-4 SCAs and Conjugates

| | | K_{off} | | |
|---|---|---|---|---|
| | Kₒₙ (1/Ms) | (1/s) | KA (1/M) | KD (M) |
| 2-7-SC-4 native | 2.72e5 | 4.95e−4 | 5.49e8 | 1.82e−9 |
| 2-7-SC-4 40K-PEG | 1.12e4 | 4.04e−6 | 2.78e9 | 3.6e−10 |

### Example 8

Assay for Neutralization of TNF-α Cellular cytotoxicity

A cell-based assay for neutralization of TNF-α cellular cytotoxicity was conducted as follows.

WEHI-13VAR cells (obtained from the American Type Culture collection, ATCC No. CRL-2148) are more sensitive to TNF-α in the presence of Actinomycin D, and were employed in the assay.

WEHI-13VAR cells were seeded in a 96-well plate, 10,000 cells per well and incubated overnight at 37° C. in a humidified incubator with 5% CO₂. A range of concentrations of D2E7 SCA proteins and their PEGylated forms were added to the seeded cells in the 96-well plates in serial dilutions from 10 g/ml to 2.5 ng/ml diluted in culture medium.

Immediately following the addition of D2E7 SCA compounds, rhTNF-alpha (Pierce) was added to each well at a concentration of 1.0 ng/ml. The cells were then allowed to grow for 24 h and cell viability was determined by addition of 15 μl MTT dye reagent (Cat # G4000, Promega Corporation [Madison, Wis.]) (3-(4,5,dimethylthiazol2yl)2,5-diphenyl tetrazolium bromide) following the manufacturer's instruction. The analysis of cell rescue was performed by comparing the viability of D2E7-treated cells with untreated cells in the presence of TNF-α.

Control wells consisted of untreated cells, and cells treated with TNF-α alone. The cells in the control wells exhibited a complete loss of viability. The percentage of viable cells (or rescued cells) in experimental wells was plotted against the log of D2E7 concentrations and IC₅₀ values were determined for each data set. Each value was derived from a triplicate experimental set.

Cell Rescue by D2E7 SCA Proteins from TNF-Alpha Lethality

The ability of the anti-TNFα SCA proteins, such as those listed by Tables 9A, 9B and 9C, infra, to protect cells from negative effects of TNFα was confirmed by employing a TNFα—sensitive cell-line, and contacting the cells with TNFα, with and without SCA protein 2-7-SC-2. Results are were consistent with additional tests conducted with other SCA proteins prepared by Example 1.

Materials and Methods

Cell line: WEHI-13VAR cells; ATCC # CRL-2148, mouse cell line Propagation: RPMI 1640 medium with 2 mM G-glutamine, 1.5 g/L sodium bicarbonate, 4.5 g/L glucose, 10 mM HEPES, and 1.0 mM sodium pyruvate, and 10% FBS Freeze Medium: Culture medium, 95% and DMSO, 5%

Assay Method

WEHI-13VAR cells were trypsinized and seeded in 96-well plate, 10,000 cells/well in complete RPMI-1640 medium and allowed to establish for 12 h in a humidified incubator at 37° C. with 5% CO₂.

Cells were washed with PBS and fresh medium was added to each well Different D2E7 SCA variants and their PEGylated forms were added to wells, including those listed by Tables 9A, 9B and 9C, infra. The SCAs were serially diluted from 10 g/ml to 2.5 ng/ml (diluted in complete RPMI medium). No D2E7 was added to the control cells. Immediately following the addition of the D2E7 compound, recombinant hTNF-alpha (1.0 ng/ml diluted in RPMI medium, not complete medium) was added to each well. No TNF-alpha was added to the untreated control cells.

Cells were incubated for 24 h in humidified incubator at 37° C. with 5% CO₂

At the end of incubation period, 15 μl MTT dye reagent (Cat # G4000, Promega Corporation) was added to each well and the plate was incubated for 4 h at 37° C. before stop solution was added to each well. The content of each well was mixed thoroughly and crystals were allowed to solubilize overnight at room temperature.

The plate was read at 570 nm and 630 nm in a 96-well plate reader (Molecular Devices) and the difference (measure of cell viability) in absorbance units was plotted against the concentration of D2E7 compound used to rescue the cells against TNF-alpha cytotoxicity. The concentration of D2E7 SCA protein at which 50% cells were rescued was determined for each set of experiment from the viability graph using log[D2E7] as X-axis and % Rescued as Y-axis parameters.

The stability of Mal-PEG (20 kDa) polymer at 25° C., 50 mM sodium phosphate pH 7.0, 1 mM EDTA was investigated by UV absorbance scanning from 220 nm to 400 nm for 0, 2, 4, 22, and 33 hours. After 33 hours at 25° C., 3 mM cysteine was added and the mixture was scanned after 5 minutes incubation. The time dependent conversion of the distinctive peak at 300 nm was quantitated.

Note: WEHI-13VAR cells are more sensitive to TNF-alpha and lymphotoxin than L929 (ATCC CCL-1). In the absence of Actinomycin D these cells lose sensitivity to TNF within 30 days. Also, addition of Actinomycin D was found to be detrimental for the rescue of cells by D2E7 compounds.

The native 2-7-SC-2, 2-7-SC-2-NE-maleimide, 5K, 20K, and 40K PEGylated 2-7-SC-2 s were analyzed for their potency to rescue cells from TNF-mediated killing. Table 9A provides the IC₅₀ values (to rescue 50% WEHI-13VAR cells from killing by 1.0 ng/ml TNF) for each version of 2-7-SC-2.

TABLE 9A
Cell rescue by 2-7-SC-2 SCA compounds

| 2-7-SC-2 Versions | IC₅₀ values |
|---|---|
| 2-7-SC-2 native | 3.05×10−9 M |
| 2-7-SC-2-NE-maleimide | 3.71×10−9 M |
| 2-7-SC-2-5K-PEG | 4.27×10−9 M |
| 2-7-SC-2-20K-PEG | 3.52×10−9 M |
| 2-7-SC-2-40K-PEG | 11.09×10−9 M |

TABLE 9B
Cell rescue by 2-7-SC-4 SCA compounds

| 2-7-SC-4 Versions | IC₅₀ Values |
|---|---|
| 2-7-SC-4 native | 7.18×10−9 M |
| 2-7-SC-4-40K-PEG | 4.64×10−9 M |

TABLE 9C
Cell rescue by 2-7-SC-5 SCA compounds

| 2-7-SC-5 Versions | IC₅₀ Values |
|---|---|
| 2-7-SC-5 native | 4.18×10−9 M |
| 2-7-SC-5-40K-PEG | 6.91×10−9 M |

These data confirm that the designed PEGylated versions of D2E7 SCA display similar bioactivity in binding and neutralization of the cytokine TNF-alpha in this cell-based assay. The PEG-SCA compounds therefore are able to effectively neutralize this cytokine and prevent its binding to the TNF-alpha receptors on these cells.

### Example 9

Pharmacokinetics of D2E7 SCA and PEG-SCAs

STUDY PROTOCOL: Pharmacokinetics of SCA and PEG-SCA Conjugates in ICR Mice

Purpose of Study

This study was designed to examine the plasma pharmacokinetics of SCA D2E7 (2-7-SC-2 and 2-7-SC-5) and the PEGylated forms including PEG (5 kd), PEG(20 kd) and PEG(43 kd) conjugates in ICR mice.

Test Articles (Stored at −20° C. prior to administration)

D2E7(2-7-SC-2, 2-7-SC-5) (100% active w/w)

PEG(20 kd)-D2E7(2-7-SC-2, 2-7-SC-5) (57.4% active w/w)

PEG(43 kd)-D2E7(2-7-SC-2, 2-7-SC-5) (38.6% active w/w)

Test System

Species: ICR (Sprague Dawley Harlan) Mice

Age: 7-8 week

Gender: Female

Weight: Weight range at initiation: approximately 25 g

Animal Husbandry:

Mice were housed 5 per cage in breeder boxes at the University of Medicine and Dentistry of New Jersey (“UMDNJ”) vivarium. Cages were sized in accordance with the “Guide for the Care and Use of Laboratory Animals of the Institute of Laboratory Animal Resource”, National Research Council. Waste material was removed at a minimum of two times per week. The cages were clearly labeled with a cage card indicating study, test article, animal number, sex, and dose level. Animals were acclimated for one week prior to study initiation

Diet

The mice had access to tap water and fed commercially available lab chow ad libitum.

Sample Preparation

D2E7(2-7-SC-2, 2-7-SC-5) were diluted with PBS to 0.556 mg/mL D2E7

PEG(20 kd)-D2E7(2-7-SC-2, 2-7-SC-5) were diluted with PBS to 0.503 mg/mL D2E7 equivalents

PEG(43 kd)-D2E7(2-7-SC-2, 2-7-SC-5) were diluted with PBS to 0.541 mg/mL D2E7 equivalents

Phosphate Buffered Saline; 10 mM sodium phosphate, pH 6.5 containing 140 mM NaCl

Administration Site

D2E7(2-7-SC-2, 2-7-SC-5), PEG(20 kd)-D2E7(2-7-SC-2, 2-7-SC-5), and PEG(43 kd)-D2E7(2-7-SC-2, 2-7-SC-5) conjugates were administered as a single dose (Day 1) i.v. via the tail vein.

Experimental Design

Fifty-four (54) mice were assigned, dosed and bled according to the scheme of Table 10,

TABLE 10

| | | | | | | | |
|---|---|---|---|---|---|---|---|
| | | | | D2E7 | | | |
| | | | Dose | Dose* | | | Vol^{§} |
| Group | Tx | N | (mg/kg) | (mg/kg) | Inj | Time Points Bled (h) | (μl) |

| | | | | | | | | | | | | | | | |
|---|---|---|---|---|---|---|---|---|---|---|---|---|---|---|---|
| 1 | 20kd | 3 | 7.0 | 4 | iv | 0.03 | | | | | | 24 | | | 100/1000 |
| | PEG-2- | | | | | | | | | | | | | | |
| | 7-SC-5 | | | | | | | | | | | | | | |
| 2 | 20kd | 3 | 7.0 | 4 | iv | | 0.25 | | | | | | 48 | | 100/1000 |
| | PEG-2- | | | | | | | | | | | | | | |
| | 7-SC-5 | | | | | | | | | | | | | | |
| 3 | 20kd | 3 | 7.0 | 4 | iv | | | 0.5 | | | | | | 72 | 100/1000 |
| | PEG-2- | | | | | | | | | | | | | | |
| | 7-SC-5 | | | | | | | | | | | | | | |
| 4 | 20kd | 3 | 7.0 | 4 | iv | | | | 1 | | | | | 96 | 100/1000 |
| | PEG-2- | | | | | | | | | | | | | | |
| | 7-SC-5 | | | | | | | | | | | | | | |
| 5 | 20kd | 3 | 7.0 | 4 | iv | | | | | 3 | | | | | 1000 |
| | PEG-2- | | | | | | | | | | | | | | |
| | 7-SC-5 | | | | | | | | | | | | | | |
| 6 | 20kd | 3 | 7.0 | 4 | iv | | | | | | 6 | | | | 1000 |
| | PEG-2- | | | | | | | | | | | | | | |
| | 7-SC-5 | | | | | | | | | | | | | | |
| 7 | 2-7-SC-5 | 3 | 4.0 | 4 | iv | 0.03 | | | | | | 24 | | | 100/1000 |
| 8 | 2-7-SC-5 | 3 | 4.0 | 4 | iv | | 0.25 | | | | | 48 | | | 100/1000 |
| 9 | 2-7-SC-5 | 3 | 4.0 | 4 | iv | | | 0.5 | | | | | | 72 | 100/1000 |
| 10 | 2-7-SC-5 | 3 | 4.0 | 4 | iv | | | | 1 | | | | | 96 | 100/1000 |
| 11 | 2-7-SC-5 | 3 | 4.0 | 4 | iv | | | | | 3 | | | | | 1000 |
| 12 | 2-7-SC-5 | 3 | 4.0 | 4 | iv | | | | | | 6 | | | | 1000 |
| 7 | 43kd | 3 | 10.4 | 4 | iv | 0.03 | | | | | | 24 | | | 100/1000 |
| | PEG-2-7- | | | | | | | | | | | | | | |
| | SC-5 | | | | | | | | | | | | | | |
| 8 | 43kd | 3 | 10.4 | 4 | iv | | 0.25 | | | | | | 48 | | 100/1000 |
| | PEG-2-7- | | | | | | | | | | | | | | |
| | SC-5 | | | | | | | | | | | | | | |
| 9 | 43kd | 3 | 10.4 | 4 | iv | | | 0.5 | | | | | | 72 | 100/1000 |
| | PEG-2-7- | | | | | | | | | | | | | | |
| | SC-5 | | | | | | | | | | | | | | |
| 10 | 43kd | 3 | 10.4 | 4 | iv | | | | 1 | | | | | 96 | 100/1000 |
| | PEG-2-7- | | | | | | | | | | | | | | |
| | SC-5 | | | | | | | | | | | | | | |
| 11 | 43-kd | 3 | 10.4 | 4 | iv | | | | | 3 | | | | | 1000 |
| | PEG-2-7- | | | | | | | | | | | | | | |
| | SC-5 | | | | | | | | | | | | | | |
| 12 | 43kd | 3 | 10.4 | 4 | iv | | | | | | 6 | | | | 1000 |
| | PEG-2-7- | | | | | | | | | | | | | | |
| | SC-5 | | | | | | | | | | | | | | |

*D2E7 equivalent
^{§}Repeat bleeding was ˜1000 μL

Two (2) untreated mice were bled via cardiac puncture into EDTA containing tubes for the collection of untreated control plasma.

Mice were injected intravenously with 200 μL per mouse with D2E7(2-7-SC-5), 180 μL per mouse PEG(20 kd)-D2E7(2-7-SC-5), and 190 μL per mouse PEG(43 kd)-D2E7(2-7-SC-5) conjugates. Following sedation with 0.09% avertin, mice were bled via the retro-orbital sinus into EDTA containing vials. At 2 min, 15 min, 30 min and 1 hour mice were bled 100 μL and at 3 h, 6 h, 24 h, 48 h, 72 h and 96 h mice were terminally bled ˜1000 μL by cardiac puncture. The plasma was collected following centrifugation of the blood and immediately frozen at −80° C. on dry ice.

The plasma samples were thawed and the concentration of D2E7 compounds determined by ELISA. The data were modeled using WinNonlin software to determine D2E7(2-7-SC-2, 2-7-SC-5), PEG(20 kd)-D2E7(2-7-SC-2, 2-7-SC-5), and PEG(43 kd)-D2E7(2-7-SC-2, 2-7-SC-5) pharmacokinetic parameters.

Clinical Examinations: The mice were examined visually on arrival. A detailed physical examination for signs of clinical abnormality was performed only when necessary according to visual assessment, in order to avoid excessive handling. The mice were examined visually once daily following infusion of the test article, for mortality and signs of reaction to treatment. Any death and clinical signs were recorded. More frequent examinations were performed if circumstances dictate.

Animal Care Provision: This study was conducted in accordance with the current guidelines for animal welfare (NIH Publication 86-23, 1985).

Pharmacokinetics of D2E7 SCA and PEG-D2E7 SCA Conjugates in Mice:

Enzyme-Linked Immunosorbent Assay (ELISA) of D2E7 SCA and PEG-SCA

Sample Preparation. The linear range of SCA tested was between 0.2 ng/ml and 30 ng/ml. For PEG-SCA, the larger the size of PEG, the less sensitivity of ELISA. A standard curve (point to point, exponential, or quadratic) was fitted to 1-100 ng/ml protein concentrations and an optical reading within the linear range was used for analysis. The standard SCA or PEG-SCA was diluted in plasma to a similar dilution factor to the plasma samples analyzed or directly diluted in dilution buffer (0.1% BSA and 0.05% Tween-20 in PBS, pH 7.4). To simplify the procedure, the standard was diluted in dilution buffer for plasma sample analysis. The dosage by i.v. or s.c. administration was 4.5 mg/kg. The dilution factors for plasma samples by i.v. administration were 500 for 0.03-3 hr samples and 10 for 6-96 hr samples of SCA, 500 for 0.03-24 hrs samples and 10 for 4-96 hrs samples of PEG-5k-SCA, 800 for 0.033-6 hr samples and 100 for 24-96 hr samples of PEG-20k-SCA, and 800 for all samples of PEG-40k-SCA. The dilution factors of the plasma samples by s.c. administration were 200 for all samples of SCA and 300 for all samples of PEG(20k)-SCA.

ELISA Procedure. A sandwich ELISA was used to determine plasma concentrations of SCA and PEG-SCA conjugates. The samples were measured in terms of defined compositions as detected by the antibody. The capture antibody was polyclonal anti D2E7 antibody which was purified by protein A and D2E7-conjugated affinity columns. For the binding to TNFα, the plate was coated with TNFα. The primary and secondary antibodies were biotinylated anti D2E7 antibody and Streptavidin-peroxidase respectively. The Maxisorp plates were coated with 400 ng/well anti D2E7 antibody or TNFα in 50 ul of 50 mM sodium bicarbonate at 25° C. for overnight. At the same time, for samples dilution, the Nunc microwell plates or any regular 96-well plates that have a minimal absorption of protein were blocked with blocking buffer (1% BSA, 5% Sucrose, and 0.05% NaN₃ in PBS, pH 7.4) at 4° C. for overnight. On the next day, the coating solution and blocking solution were removed from both ELISA and Nunc plates with aspirator. The ELISA plates were blocked with blocking solution (250 ul/well) for at least 1 hr at 25° C. and the Nunc plates were washed three times with wash buffer (PBS with 0.05% Tween-20, pH 7.4) or were allowed to air dry at 25° C. and stored at 4° C. for further analysis. The ELISA plates after removing blocking solution were washed with wash buffer three times or allowed to air dry at 25° C. and stored sealed at 4° C. until further use. The plasma samples were diluted 1:2 in a consecutive manner from the top of the Nunc plate to the bottom with 120 ul left in each well. After the pre-dilution with dilution buffer, 100 ul of the samples was transferred to ELISA plates and incubated at 4° C. overnight. After the sample solutions were removed and the plates were washed three times with wash buffer, 20-ng biotin anti D2E7 antibody in 50 ul dilution buffer was added to each well. The samples were incubated at 25° C. for 2 hrs. 100-ul streptavidin-peroxidase was added at 1: 16,000 dilution after the primary antibody was removed and the plates were washed with wash buffer for four times. The plates were incubated at 25° C. for 1 hr. The solution was removed and the plates were washed three times with wash buffer. The color was developed 10-20 min after adding 100 ul of TMBE substrate and stopped by adding 50 ul 1 M H₂SO₄. Absorbance at 450 nm was recorded.

Data Acquisition and Analysis. Data were acquired and analyzed on a Molecular Devices microplate reader. The standard curve was obtained by plotting of optical density of the endpoints at 450 nm versus the concentrations of the standard and by drawing the best fitting curve that has a correlation coefficient of 0.99 or better. All unknown sample concentrations were calculated from the standard curve after the dilution factor has been incorporated. The closest numbers of all data points that have an appropriate optical density have been averaged for the results.

Pharmacokinetics of D2E7 SCA and PEG-D2E7 SCA Conjugates in Mice

PK parameters for all 2-7-SC-2 series (2-7-SC-2, ethyl-2-7-SC-2, PEG-5k-2-7-SC-2,

PEG-20k-2-7-SC-2, PEG-40k-2-7-SC-2) were determined.

PK parameters for 2-7-SC-5, PEG-20k-2-7-SC-5, and PEG-40k-2-7-SC-5 were determined.

PK parameters for 2-7-SC-2 and PEG-20k-2-7-SC-2 by s.c. injection were also determined.

From the pilot experiments, a lower detection sensitivity of 2-7-SC-2 (50 ng/ml) using anti 218 linker was observed.

An approximately 100-fold extension of circulating half-life in mice was observed in the 40 kDa PEG-SCA compounds (2-7-SC-2, 2-7-SC-5) when compared to the unmodified SCA protein.

Table 11, below, displays the determined pharmacokinetic parameters for 2-7-SC-2, PEG-2-7-SC-2, 2-7-SC-5, PEG-2-7-SC-5 administered via intravenous injection (IV) or subcutaneous (SC) injection. (Table 11 on separate page)

TABLE 11
Pharmacokinetic Parameters of D2E7 SCA and PEG-SCA in Mice¹

| SCA/ | | | | | AUC | CL | Vss | Cₘₐₓ |
|---|---|---|---|---|---|---|---|---|
| PEG | Rte | _{1/2} (hr) | _{1/2} (hr) | MRT (hr) | hr.μg/ml | ml/hr/kg | ml/kg | μg/ml |
| 2-7-SC2 | i.v. | 0.15 ± 0.03 | 0.70 ± 0.20 | 0.37 ± 0.23 | 11.3 ± 1.4 | 387 ± 49 | 280 ± 57 | 53.8 ± 3.8 |
| Ethyl- | i.v. | 0.08 ± 0.00 | 0.7 ± 00 | 0.44 ± 0.02 | 4.2 ± 0.1 | 1035 ± 12 | 450 ± 20 | 36.5 ± 0.1 |
| 2-7-SC2 | | | | | | | | |
| PEG(5k)- | i.v. | 2.57 ± 0.87 | 6.74 ± 5.19 | 8.21 ± 6.23 | 338 ± 80 | 10.5 ± 2.6 | 84.1 ± 33.7 | 104 ± 6 |
| 2-7-SC2² | | | | | | | | |
| PEG(20k) | i.v. | 4.38 ± 1.76 | 27.2 ± 49.0 | 19.9 ± 34.1 | 347 ± 138 | 12.6 ± 5.0 | 250 ± 346 | 54.9 ± 1.4 |
| 2-7-SC2³ | | | | | | | | |
| PEG(40k) | i.v. | 21.62 ± 2.64 | 28.3 ± 4.1 | 40.6 ± 5.8 | 3463 ± 387 | 1.26 ± 0.14 | 51.3 ± 3.2 | 111 ± 5 |
| 2-7-SC2 | | | | | | | | |
| 2-7-SC-5 | i.v. | 0.23 ± 0.01 | 1.34 ± 0.13 | 1.51 ± 0.16 | 19.1 ± 1.0 | 203 ± 10 | 308 ± 18 | 57.8 ± 0.9 |
| PEG(20k) | i.v. | 3.57 ± 0.97 | 7.00 ± 2.64 | 9.79 ± 3.66 | 449.5 ± 115.1 | 8.91 ± 1.46 | 81.0 ± 10.3 | 88.2 ± 10.8 |
| EN456² | | | | | | | | |
| PEG(40k) | i.v. | 20.7 ± 3.8 | 30.7 ± 14.0 | 41.2 ± 15.3 | 2624 ± 466 | 1.36 ± 0.24 | 56.0 ± 13.5 | 88.0 ± 3.6 |
| -2-7-SC5 | | | | | | | | |
| 2-7-SC2 | s.c. | 1.4 ± 30.2³ | | | 22.4 ± 5.0 | 178 ± 39 | | 4.11 ± 0.44 |
| PEG(20k) | s.c. | 26.0 ± 3.0 | | | 1925 ± 115 | 2.08 ± 0.12 | | 39.0 ± 1.8 |
| 2-7-SC2 | | | | | | | | |

¹Pharmacokinetic parameters for intravenous (“i.v.”) were determined by using a two compartment, i.v. bolus, no lag time, 1st order elimination model. Pharmacokinetic parameters for sc were determined by using a one compartment, 1st order input, 1st order elimination model.
²The data are an average of two analyses of computer modeling and the highest standard deviation was taken.
³High numbers of standard deviation.

These results demonstrate that the circulating lives of PEGylated SCA proteins can designed to cover the range of therapeutically useful pharmacokinetics. The two-log extension of serum half-life in the 40 kDa PEG conjugated SCA places these compounds in the pharmacokinetic range of intact monoclonal antibodies. The site-specific attachment of the PEG polymer at a unique site distant from the antigen-binding site allows the manufacture of not only active antigen-binding proteins, but also production of a product that is relatively homogeneous in its composition, in contrast to the substantial heterogeneity of SCA proteins PEGylated using random amine chemistries. The large difference in circulating lives of the 40 kDa PEGylated SCA proteins of this study when compared to the 20 kDa PEGylated SCA proteins was somewhat surprising. We did not predict this based on our results of randomly PEGylated (CC49) SCA proteins, where there was evidence of a pharmacokinetic plateau at about 20 kDa PEG, since the 12 kDa PEG displayed comparable circulating lives.

While not being bound by any theory or hypothesis as to how the invention may operate, it is believed that it is the branched chain structure that also contributes to vastly prolonged circulating lives of the 40 kDa PEG-SCA compounds. Of special interest is the success in using the PEG-SCA (20 kDa PEG) in subcutaneous injections. This route of administration provided markedly better AUC values than intravenous administration. The subcutaneous route may ultimately be preferred for the formulation of PEG-SCA therapeutics. The linker attachment of PEG to the SCA proved successful and compared well in animal studies with the C-terminal PEG attachment. Possibly, the linker attachment of PEG could also contribute to promotion of SCA stability and diminished antigenicity and/or proteolysis.

### Example 10

Dimeric D2E7 PEG-SCA Proteins VIA Bis-Maleimide-PEG

In order to generate bivalent PEG-SCA compounds having two SCA proteins per one polymer, bis-maleimide-PEG polymers were employed. These have the activated maleimide group at both termini of the polymer. SDS PAGE analysis demonstrated that the SCA-PEG-SCA compounds could be synthesized using the methods of this disclosure. The effects of reaction pH and the reaction molar ratio are shown in Table 12 and Table 13, respectively.

Effect of Reaction Molar Ratio on Formation of Bis- and Mono-D2E7 SCA—

TABLE 12

| bis-mal-PEG:D2E7 EN450 | 0.165:1 | 0.335:1 | 1:1 |
|---|---|---|---|
| bis-D2E7 SCA-PEG conjugate (%) | 20.7 | 21.5 | 17.6 |
| mono-D2E7 SCA-PEG conjugate (%) | 9.4 | 21.3 | 26.0 |
| bis- and mono-D2E7-SCA-PEG conjugates | 30.1 | 42.8 | 43.6 |
| (%) | | | |

The data were obtained by gel image analysis on 4-20% SDS-PAGE gel. Bis-mal-PEG (20 k) was dissolved in 100 mM sodium phosphate, pH 6 to a concentration of 3.7 mg/ml. It was then slowly added to 1 mg/ml D2E7 EN450 in 100 mM sodium phosphate, pH 6 and 1 mM EDTA at 1/30 to 1/10 volume of D2E7 EN450 and the reaction molar ratio indicated. The reaction was conducted at 25° C. under Nitrogen for 1.5 hrs.

Effect of Reaction pH on Formation of Bis- and Mono-D2E7 SCA-PEG (20k)

TABLE 13

| pH | 5.0 | 5.5 | 6.0 | 6.5 | 7.0 | 7.5 |
|---|---|---|---|---|---|---|
| bis-D2E7 EN450-PEG (%) | 17.6 | 19.0 | 28.2 | 34.0 | 37.7 | 38.4 |
| mono-D2E7 EN450-PEG (%) | 21.4 | 23.9 | 13.7 | 18.2 | 14.5 | 16.4 |
| bis- and mon-D2E7 EN450- | 39.0 | 42.9 | 41.9 | 52.1 | 52.1 | 54.9 |
| PEG (%) | | | | | | |

The reaction contained 1 mg/ml D2E7 EN450 and 0.12 mg/ml bis-mal-PEG compound at a reaction molar ratio of 0.165:1 (bis-mal-PEG:EN450) in 100 mM sodium phosphate, at the pH indicated. The reaction was conducted at 25° C. , under Nitrogen for 2 hrs. The samples were analyzed on 4-20% SDS-PAGE gel and the corresponding band of each compound were quantitated. High molecular weight impurities were less than 1% and the dimer of EN450 was less than 5%.

What is claimed is:

1. A single-chain antigen-binding polypeptide capable of site-specific conjugation to a polyalkylene oxide polymer, that comprises,
(a) a first polypeptide comprising an antigen-binding portion of a variable region of an antibody heavy or light chain;
(b) a second polypeptide comprising an antigen-binding portion of a variable region of an antibody heavy or light chain; and
(c) a peptide linker linking the first and second polypeptides,
wherein the single-chain antigen-binding polypeptide has at least one Cys residue which is capable of being conjugated to a polyalkylene oxide polymer, and has at least one antigen binding site, and
wherein the Cys residue is located at a position selected from the group consisting of:
(i) a C-terminus of the heavy chain or light chain variable region;
(ii) an N-terminus of the heavy chain or light chain variable region;
(iii) any amino acid position of the peptide linker;
(iv) both the N-terminus and C-terminus;
(v) position 2 of the linker;
(vi) both position 2 of the linker and the C-terminus; and
(iv) combinations thereof;

2. The single-chain antigen-binding polypeptide of claim 1 wherein the Cys residue is located at a position selected from the group consisting of position 2 of the linker, the C-terminus and combinations thereof.

3. The single-chain antigen-binding polypeptide of claim 1 wherein the first polypeptide comprises a variable region of an antibody light chain and the second polypeptide comprises a variable region of an antibody heavy chain.

4. The single-chain antigen-binding polypeptide of claim 1, wherein the C-terminus of the second polypeptide is the native C-terminus.

5. A conjugate comprising the single-chain antigen-binding polypeptide of claim 1, and comprising a polyalkylene oxide polymer, wherein the polyalkylene oxide polymer is covalently linked to the single-chain antigen-binding polypeptide at a Cys residue.

6. The conjugate of claim 5 wherein the polyalkylene oxide is linked to the single-chain antigen-binding polypeptide at a Cys residue via a linker selected from the group consisting of a maleimide, vinylsulfone, thiol, orthopyridyl disulfide and a iodoactemide linker.

7. The conjugate of claim 5 wherein the polyalkylene oxide is linked to the single-chain antigen-binding polypeptide at a Cys residue via a maleimide linker.

8. The conjugate of claim 5 wherein the polyalkylene oxide ranges in size from about 5,000 to about 40,000 Daltons.

9. The conjugate of claim 5 wherein the polyalkylene oxide is a polyethylene oxide.

10. The conjugate of claim 5, wherein the polyalkylene oxide is conjugated to at least two single-chain antigen-binding polypeptides, and each single-chain antigen-binding polypeptide is the same, or different.

11. The conjugate of claim 10 wherein the single-chain antigen-binding polypeptide is further conjugated to an additional functional moiety.

12. The conjugate of claim 11 wherein the additional functional moiety is a detectable label or tag.

13. A polynucleotide encoding the single-chain antigen-binding polypeptide of claim 1.

14. A replicable expression vector comprising the polynucleotide of claim 13.

15. A method of producing the single-chain antigen-binding polypeptide, comprising the steps of:
(a) culturing a host cell comprising the expression vector of claim 12, and
(b) collecting the single-chain antigen-binding polypeptide expressed by the host cell.

16. A method of detecting an antigen suspected of being in a sample, comprising:
(a) contacting the sample with a reagent comprising the single-chain antigen-binding polypeptide of claim 1, and
(b) detecting whether the single-chain antigen-binding polypeptide has bound to the antigen.

17. A method of detecting an antigen suspected of being in a sample, comprising
(a) contacting the sample with a reagent comprising the conjugate of claim 5, and wherein the conjugate polypeptide is conjugated to a polyalkylene oxide polymer and
(b) detecting whether the conjugate or the single-chain antigen-binding polypeptide has bound to the antigen.

18. The method of claim 17 wherein the conjugate is anchored to a solid substrate.

19. A method of treating or diagnosing a disease or disorder in a mammal in need thereof, comprising administering an effective amount of the single-chain antigen-binding polypeptide of claim 1, wherein the single-chain antigen-binding polypeptide binds to an antigen that is related to treating or diagnosing the disease or disorder.

20. A method of treating or diagnosing a disease or disorder in a mammal in need thereof, comprising administering an effective amount of the conjugate of claim 5, wherein the single-chain antigen-binding polypeptide binds to an antigen that is related to treating or diagnosing the disease or disorder.

21. A protein comprising two or more single-chain antigen-binding polypeptides capable of site-specific conjugation to at least one polyalkylene oxide polymer, wherein each single-chain antigen-binding polypeptide comprises,
(a) a first polypeptide comprising an antigen binding portion of a variable region of an antibody heavy or light chain;
(b) a second polypeptide comprising an antigen binding portion of a variable region of an antibody heavy or light chain; and
(c) a peptide linker linking the first and second polypeptides,
wherein at least one of the single-chain antigen-binding polypeptides has from 1 to 4 Cys residues capable of being conjugated to a polyalkylene oxide polymer, and wherein each single-chain antigen-binding polypeptide has at least one antigen binding site, and
wherein the Cys residue is located at a position of each Cys-bearing single-chain antigen-binding polypeptide that is selected from the group consisting of:
(i) a C-terminus of the heavy chain or light chain variable region;
(ii) an N-terminus of the heavy chain or light chain variable region;
(iii) any amino acid position of the peptide linker;
(iv) both the N-terminus and C-terminus;
(v) position 2 of the linker;
(vi) both position 2 of the linker and the C-terminus; and
(iv) combinations thereof;
wherein each single-chain antigen-binding polypeptide is the same or different.

22. The protein of claim 21 that is bivalent or trivalent.

23. The protein of claim 21 wherein the constituent single-chain antigen-binding polypeptides are noncovalently associated.

24. The protein of claim 23 wherein the peptide linker of the constituent single-chain antigen-binding polypeptides ranges in size from 2 to 18 residues.

25. The protein of claim 19 wherein the constituent single-chain antigen-binding polypeptides are covalently linked.

26. The protein of claim 21 that is encoded as a single, multivalent protein.

27. A polynucleotide encoding the protein of claim 26.
